(19) United States
(12) Patent Application Publication
(10) Pub. No.: US 2026/0216113 A1
(43) Pub. Date: Jul. 30, 2026

(54) COMPOSITIONS AND METHODS FOR DIAGNOSIS AND TREATMENT OF CONDITIONS RELATED TO AGING

(71) Applicant: Epitracker, Inc., San Diego, CA (US)

(72) Inventor: Stephanie VENN-WATSON, San Diego, CA (US)

(21) Appl. No.: 19/321,031

(22) Filed: Sep. 5, 2025

Related U.S. Application Data
(63) Continuation of application No. 17/086,198, filed on Oct. 30, 2020, now abandoned.
Application No. 17/086,198 is a continuation of application No. PCT/US2019/032274, filed on May 14, 2019.
(60) Provisional application No. 62/672,145, filed on May 16, 2018.
(60) Provisional application No. 62/838,249, filed on Apr. 24, 2019.

(51) Int. Cl. A61K 31/20; A61P 39/00

(52) U.S. Cl. CPC A61K 31/20; A61P 39/00

(57) ABSTRACT

Compositions including odd chain saturated fatty acids, and salts and derivatives thereof, and methods for treatment or prophylaxis of conditions related to aging are provided, including compositions and methods for treating conditions related to aging, including hypercholesterolemia, thrombosis, fibrosis, wound healing, hyperglobulinemia, and hypersensitivity disorder.

### INCORPORATION BY REFERENCE TO RELATED APPLICATIONS

This application claims the benefit of U.S. Provisional Application No. 62/672,145, filed May 16, 2018, and U.S. Provisional Application No. 62/838,249, filed Apr. 24, 2019. Each of the aforementioned applications is incorporated by reference herein in its entirety, and each is hereby expressly made a part of this specification.

### FIELD OF THE INVENTION

Compositions including odd chain saturated fatty acids, and salts and derivatives thereof, and methods for treatment or prophylaxis of conditions related to aging are provided, including compositions and methods for treating conditions related to aging, including hypercholesterolemia, thrombosis, fibrosis, wound healing, hyperglobulinemia, and hypersensitivity disorders.

### BACKGROUND OF THE INVENTION

Aging increases the risk of health conditions that can decrease quality of life and longevity. As people age, they have a higher risk of developing a suite of conditions, including hypercholesterolemia, thrombosis, fibrosis, wound healing, hyperglobulinemia, and hypersensitivity disorders. Aging has been identified as a causative or contributing factor to these conditions, and as such, treatments of these conditions have been proposed as a means to improve longevity and quality of life.

### SUMMARY OF THE INVENTION

Compositions and methods for treatment or prophylaxis of aging-associated conditions are provided. These compositions comprise one or more odd chain saturated fatty acids, derivatives of odd chain saturated fatty acids, or salts thereof, which may be administered in combination with other medicaments or as part of various treatment regimens as described herein. The provided compositions are effective for modulating markers of aging-associated conditions. Methods are provided for administering the compositions. These compositions may be administered in combination with other medicaments or as part of various treatment regimens as described herein. The provided compositions are effective for modulating markers of aging-associated conditions that impact quality of life or longevity. Methods are provided for administering the compositions.

The compositions are suitable for the treatment, amelioration, or prevention of conditions including but not limited to Th1-type inflammation, Th2-type inflammation, T-cell dependent B cell proliferation, allergy, asthma, atherosclerosis, autoimmunity, chronic inflammation, chronic obstructive pulmonary disease (COPD), Crohn's disease, cutaneous responses to tissue damage, fibrosis, hematological oncology, metabolic diseases, organ transplantation, psoriasis, pulmonary fibrosis, pulmonary responses to respiratory infections, restenosis, rheumatoid arthritis, sarcoidosis, stromal biology in tumors, systemic lupus erythematosus (SLE), ulcerative colitis, and vascular inflammation.

Diseases that are driven or exacerbated by the following factors may be attenuated or treated by compositions as disclosed herein: alpha smooth muscle actin (αSMA), CD40, CD69, collagen I, collagen III, decorin, e-selectin, eotaxin 3 (CCL26), fibroblast proliferation, human leukocyte antigen-DR isotype (HLA-DR), immunoglobulin G, interferon gamma-induced protein 10 (IP-10/CXCL10), interferon-inducible T cell alpha chemoattractant (I-TAC/CXCL11), interleukin (IL)-1, IL-1α, IL-2, IL-6, IL-8 (CXCL8), IL-10, IL-17A, IL-17F, keratin 8/81, macrophage colony-stimulating factor (M-CSF), matrix metalloproteinase (MMP)-1, MMP-9, monocyte chemoattractant protein 1 (MCP-1), monokine induced by gamma interferon (MIG/CXCL9), plasminogen activation inhibitor 1 (PAI-1), prostaglandin E2 (PGE2), serum amyloid A, T or B cell proliferation, tissue plasminogen activator (tPA), tumor necrosis factor alpha (TNFα), vascular cell adhesion molecule (VCAM-1), vascular endothelial growth factor 2 (VEGFR2).

Accordingly, in a generally applicable first aspect (i.e., independently combinable with any of the aspects or embodiments identified herein), a pharmaceutical composition for treatment of a condition related to aging is provided, wherein the condition related to aging is selected from the group consisting of hypercholesterolemia, thrombosis, fibrosis, wound healing, hyperglobulinemia, and hypersensitivity disorders, the composition comprising: one or more odd chain saturated fatty acids, or pharmaceutically acceptable salts thereof, wherein the one or more fatty acids are selected from the group consisting of odd chain saturated fatty acids; and a pharmaceutically acceptable carrier.

In an embodiment of the first aspect (i.e., independently combinable with any of the aspects or embodiments identified herein), the one or more fatty acids is heptadecanoic acid or pentadecanoic acid.

In an embodiment of the first aspect (i.e., independently combinable with any of the aspects or embodiments identified herein), the composition is substantially free from even chain saturated fatty acids.

In an embodiment of the first aspect (i.e., independently combinable with any of the aspects or embodiments identified herein), the composition is substantially free from polyunsaturated fatty acids.

In an embodiment of the first aspect (i.e., independently combinable with any of the aspects or embodiments identified herein), the composition is in a unit dosage form.

In an embodiment of the first aspect (i.e., independently combinable with any of the aspects or embodiments identified herein), the composition is configured for administration of from 2.5 mg to 50 mg, per 1 kg of body weight, of the one or more fatty acids or pharmaceutically acceptable salts thereof to a patient.

In an embodiment of the first aspect (i.e., independently combinable with any of the aspects or embodiments identified herein), the composition is configured for administration once per day.

In an embodiment of the first aspect (i.e., independently combinable with any of the aspects or embodiments identified herein), the composition comprises from 0.01 mg to 10000 mg of the one or more fatty acids or pharmaceutically acceptable salts thereof.

In a generally applicable second aspect (i.e., independently combinable with any of the aspects or embodiments identified herein), use is provided of a pharmaceutical composition of the first aspect or any of its embodiments in the manufacture of a medicament for treatment or prophylaxis of a condition related to aging, wherein the condition related to aging is selected from the group consisting of hypercholesterolemia, thrombosis, fibrosis, wound healing, hyperglobulinemia, and hypersensitivity disorders.

In an embodiment of the second aspect (i.e., independently combinable with any of the aspects or embodiments identified herein), the use is the manufacture of a medicament for treatment or prophylaxis of thrombosis, fibrosis, or poor wound healing.

In an embodiment of the second aspect (i.e., independently combinable with any of the aspects or embodiments identified herein), the pharmaceutical composition is configured to modulate a marker or a symptom of thrombosis, fibrosis, or poor wound healing.

In an embodiment of the second aspect (i.e., independently combinable with any of the aspects or embodiments identified herein), the marker of thrombosis, fibrosis or poor wound healing is selected from the group consisting of serum, plasma, cell, or tissue levels of odd chain saturated fatty acids, urokinase plasminogen activator, plasminogen activation inhibitor-1, or Collagen-I.

In an embodiment of the second aspect (i.e., independently combinable with any of the aspects or embodiments identified herein), the pharmaceutical composition is configured to modulate a marker or a symptom of hyperglobulinemia or hypersensitivity.

In an embodiment of the second aspect (i.e., independently combinable with any of the aspects or embodiments identified herein), the marker of hyperglobulinemia or hypersensitivity is selected from the group consisting of serum, plasma, cell, or tissue levels of odd chain saturated fatty acids, serum globulins, or immunoglobulin G.

In an embodiment of the second aspect (i.e., independently combinable with any of the aspects or embodiments identified herein), the pharmaceutical composition is configured to increase a serum, plasma, or a red blood cell membrane concentration of the one or more fatty acids to a concentration greater than 2.2 μM and less than 30 μM.

In a generally applicable third aspect (i.e., independently combinable with any of the aspects or embodiments identified herein), a method is provided for treatment or prophylaxis of a condition related to aging, wherein the condition related to aging is selected from the group consisting of hypercholesterolemia, thrombosis, fibrosis, wound healing, hyperglobulinemia, and hypersensitivity disorders, the method comprising: administering to a patient in need thereof, an effective amount of one or more fatty acids, or pharmaceutically acceptable salts thereof, wherein the one or more fatty acids are selected from the group consisting of one or more odd chain fatty acids and combinations thereof.

In an embodiment of the third aspect (i.e., independently combinable with any of the aspects or embodiments identified herein), the one or more fatty acids or pharmaceutically acceptable salts thereof is provided as a pharmaceutical composition in a unit dosage form comprising the one or more fatty acids or pharmaceutically acceptable salts thereof and a pharmaceutically acceptable carrier.

In an embodiment of the third aspect (i.e., independently combinable with any of the aspects or embodiments identified herein), the unit dosage form comprises from 0.01 mg to 10000 mg of the one or more fatty acids or pharmaceutically acceptable salts thereof.

In an embodiment of the third aspect (i.e., independently combinable with any of the aspects or embodiments identified herein), the one or more odd chain fatty acids is heptadecanoic acid or pentadecanoic acid.

In an embodiment of the third aspect (i.e., independently combinable with any of the aspects or embodiments identified herein), the pharmaceutical composition is substantially free from even chain saturated fatty acids.

In an embodiment of the third aspect (i.e., independently combinable with any of the aspects or embodiments identified herein), the pharmaceutical composition is substantially free from polyunsaturated fatty acids.

In an embodiment of the third aspect (i.e., independently combinable with any of the aspects or embodiments identified herein), the pharmaceutical composition comprises a plurality of different fatty acids.

In an embodiment of the third aspect (i.e., independently combinable with any of the aspects or embodiments identified herein), from 2.5 mg to 50 mg of the one or more fatty acids or pharmaceutically acceptable salts thereof is administered to the patient, per 1 kg of body weight, per day.

In an embodiment of the third aspect (i.e., independently combinable with any of the aspects or embodiments identified herein), the one or more fatty acids or pharmaceutically acceptable salts thereof is administered to the patient once per day.

In an embodiment of the third aspect (i.e., independently combinable with any of the aspects or embodiments identified herein), a serum, plasma, red blood cell, or tissue concentration is increased to greater than 2.2 μM and less than 30 μM.

In a generally applicable fourth aspect (i.e., independently combinable with any of the aspects or embodiments identified herein), a composition substantially as described herein is provided.

In a generally applicable fifth aspect (i.e., independently combinable with any of the aspects or embodiments identified herein), a method substantially as described herein is provided.

In a generally applicable sixth aspect (i.e., independently combinable with any of the aspects or embodiments identified herein), a use substantially as described herein is provided.

Any of the features of an embodiment of the first through sixth aspects is applicable to all aspects and embodiments identified herein. Moreover, any of the features of an embodiment of the first through sixth aspects is independently combinable, partly or wholly with other embodiments described herein in any way, e.g., one, two, or three or more embodiments may be combinable in whole or in part. Further, any of the features of an embodiment of the first through sixth aspects may be made optional to other aspects or embodiments. Any aspect or embodiment of a method or use can be performed using a composition of another aspect or embodiment, and any aspect or embodiment of a composition can be adapted to a method or use of another aspect or embodiment.

### DESCRIPTION OF THE DRAWINGS

FIG. 1. Prominent Stage 3 “bridging” liver fibrosis (black arrows) in high fat diet-induced non-alcoholic steatohepatitis in New Zealand white control rabbits compared to lack of bridging fibrosis in rabbits treated with daily oral pentadecanoic acid (35 mg/kg) for 11 weeks.

FIG. 2. Dose-dependent lowering of secreted immunoglobulin G (IgG) in CD19+ B cells and peripheral blood mononuclear cells stimulated with α-IgM and TCR ligands and incubated with pentadecanoic acid at 6.7 μM and 20 μM compared to controls (n=6).

FIG. 3. Dose-dependent increases in urokinase plasminogen activator receptor (uPAR) in primary human endothelial cells stimulated with IL-1B, TNFα, and IFNγ and incubated with pentadecanoic acid at 6.7 μM and 20 μM compared to controls (n=6).

FIG. 4. Dose-dependent lowering of Collagen-I, plasminogen activator inhibitor-1 (PAI-1), and 72-hour fibroblast proliferation in primary human dermal fibroblasts stimulated with IL-1B, TNFα, IFNγ, EGF, bFGF, and PDGF-BB and incubated with pentadecanoic acid at 6.7 μM and 20 μM compared to controls (n=6).

### DETAILED DESCRIPTION

Compositions including one or more odd chain saturated fatty acids, and associated methods for treatment of conditions related to aging, including hypercholesterolemia, thrombosis, fibrosis, wound healing, hyperglobulinemia, and hypersensitivity disorders and other related conditions, are provided.

Aging increases the risk of hypercholesterolemia (Kriesberg R A and Kasim S (1987) Cholesterol metabolism and aging, Am J Med 82:54-60). Elevated cholesterol, especially elevated low-density lipoprotein (LDL) cholesterol, have been identified as underlying causes of or contributors to cardiovascular disease, including atherosclerosis, which also increase in prevalence with age. Lower cholesterol levels, especially for people under 50 years old, have been associated with improved longevity (Anderson K M, Castelli W P, Levy D (1987) Cholesterol and mortality: 30 years of follow-up from the Framingham Study JAMA 257:2176-2180).

Aging increases the accumulation of Collagen-I and Collagen-III, resulting in progressive fibrotic diseases of the heart, lung, liver, kidney, and skin that negatively impact cardiac, respiratory, hepatic, and renal function, as well as wound healing. Increased fibrosis may be due, in part, to raised circulating PAI-1 and decreased uPA, which increase the risk of thrombosis and decrease the active breakdown of fibrotic tissue, including Collagen-I (Ghosh AK, Vaughan DE 2011 PAI-1 in tissue fibrosis J Cell Physiol 227:493-507). Agents that reduce PAI-1 and increase uPA can help treat thromboses, and by lowering Collagen-I deposition, can also help treat fibrotic diseases and improve aging-associated delayed wound healing.

Aging has been associated with higher circulating levels of autoantibodies and immunoglobulins, including immunoglobulin G (IgG) (Hallgren et al. 1973 Lymphocyte phytohemagglutinin responsiveness, immunoglobulins and autoantibodies in aging humans). IgG, the most common circulating antibody, is released by B cells as a component of humoral immunity. While IgG aids in fighting infections, it also plays a pathogenic role in type II and type III hypersensitivity reactions, including but not limited to autoimmune hemolytic anemia, serum sickness, systemic lupus erythematosus and hypersensitivity pneumonitis. Agents that lower IgG secretion by B cells can help treat hyperglobulinemia and hypersensitivity disorders.

It is an object of certain of the embodiments to provide a method for detecting protective factors for and risk factors against conditions provided herein, including but not limited to aging and associated conditions such as hypercholesterolemia, thrombosis, fibrosis, wound healing, hyperglobulinemia, and hypersensitivity disorders, and other related conditions in mammal subjects, such as companion animals and humans. As an object in certain embodiments, the compositions support healthy cholesterol, or maintain healthy platelets, or maintain a healthy allergen response. As an object in certain embodiments, the compositions are suitable for use as antiproliferative agents for dermal fibroblasts, and can assist in wound healing, especially diminishing scar formation, and can be administered systemically or topically. An object of certain of the embodiments is to provide a method for treating conditions including but not limited to aging-associated conditions in mammal subjects, such as companion animals and humans. An object of certain of the embodiments is to provide a method for detecting conditions including but not limited to aging-associated conditions in mammal subjects, such as companion animals and humans. It is an object of certain of the embodiments to provide a method for increasing the serum, plasma, or erythrocyte membrane level of one or more fatty acids or fatty acid derivatives, including but not limited to odd chain fatty acids, for example, heptadecanoic acid, and/or certain even chain fatty acids, such as behenic acid, in mammal subjects, such as companion animals and humans. An object of certain of the embodiments is to provide a fatty acid supplement or prescription therapeutic for treating or preventing conditions including but not limited to those associated with aging. An object of certain of the embodiments is to provide a method for detecting and/or treating a condition provided herein including aging-associated conditions in mammal subjects, such as companion animals and humans, that is easy to accomplish in a cost-effective manner.

An object of certain of the embodiments is to provide a method for modulating markers of aging-associated conditions in mammal subjects, such as companion animals and humans. An object of certain of the embodiments is to provide a method for detecting aging-associated conditions in mammal subjects, such as companion animals and humans. An object of certain of the embodiments is to provide a method for treatment of aging-associated conditions in mammal subjects, such as companion animals and humans. An object of certain of the embodiments is to provide a method for prophylaxis of aging-associated conditions in mammal subjects, such as companion animals and humans. An object of certain of the embodiments is to provide a method for prophylaxis of a condition provided herein including aging-associated conditions and associated conditions, including hypercholesterolemia, thrombosis, fibrosis, wound healing, hyperglobulinemia, and hypersensitivity disorders, in mammal subjects, such as companion animals and humans.

An object of certain of the embodiments is to provide a method for increasing an odd chain fatty acid in the sera, plasma, or erythrocyte membranes of mammal subjects, such as companion animals and humans. An object of certain of the embodiments is to provide a method for detecting or treating aging-associated conditions in mammal subjects, such as companion animals and humans. An object of certain of the embodiments is to provide an odd chain fatty acid substantially free from other fatty acids in mammal subjects, such as companion animals and humans. An object of certain of the embodiments is to provide one or more odd chain fatty acids substantially free from even chain fatty acids in mammal subjects, such as companion animals and humans.

It is an object of certain of the embodiments is to provide a method for detecting and treating aging-associated conditions in mammal subjects, such as companion animals and humans. An object of certain of the embodiments is to provide a fatty acid, such as an odd chain fatty acid, for treating aging-associated conditions in mammal subjects, such as companion animals and humans. An object of certain of the embodiments is to provide a method for prophylaxis of aging-associated conditions in mammal subjects, such as companion animals and humans. An object of certain of the embodiments is to provide a method for detecting or treating a condition associated with aging in mammal subjects, such as companion animals and humans. An object of certain of the embodiments is to provide pentadecanoic acid or other odd chain saturated fatty acid supplement for treating aging-associated conditions in mammal subjects, such as companion animals and humans.

An object of certain of the embodiments is to provide a bioavailable form of odd chain fatty acids to mammal subjects, such as companion animals and humans. An object of certain of the embodiments is to provide one or more odd chain fatty acids with one or more certain even chain fatty acids to mammal subjects, such as companion animals and humans. An object of certain embodiments is to provide a method for increasing both an odd chain fatty acid and certain even chain fatty acids in the sera of mammal subjects, such as companion animals and humans. An object of certain of the embodiments is to provide a method for fatty acid elongation in the sera, plasma, or erythrocyte membranes of mammal subjects, such as companion animals and humans. An object of certain of the embodiments is to provide a method for fatty acid chain shortening in the sera, plasma, or erythrocyte membranes of mammal subjects, such as companion animals and humans. An object of certain of the embodiments is to provide a method for altering concentrations of a variety of odd chain and very long even chain fatty acid forms, including neutral forms (e.g. free fatty acids, cholesterol esters, diacylglycerides, and phospholipids triacylglycerides), (e.g. phosphatidylcholine, phosphatidylethanolamine, lysophosphatidylcholine, and lysophosphatidylethanolamine), and sphingolipids (e.g. ceramides, hexosylceramides, and sphingosines) in the sera, plasma, or erythrocyte membranes of mammal subjects, such as companion animals and humans.

Compositions including one or more of certain even chain fatty acids, and associated methods for treatment of conditions associated with aging are provided. Compositions including one or more bioavailable even chain fatty acids are provided.

One or more than one of the aforementioned objects is provided by or achieved by the various compositions, methods, and uses as described herein.

### Definitions

The term “alcohol” as used herein is a broad term, and is to be given its ordinary and customary meaning to a person of ordinary skill in the art (and is not to be limited to a special or customized meaning), and refers without limitation to any compound as described herein incorporating one or more hydroxy groups, or being substituted by or functionalized to include one or more hydroxy groups.

The term “derivative” as used herein is a broad term, and is to be given its ordinary and customary meaning to a person of ordinary skill in the art (and is not to be limited to a special or customized meaning), and refers without limitation to any compound as described herein incorporating one or more derivative groups, or being substituted by or functionalized to include one or more derivative groups. Derivatives include but are not limited to esters, amides, anhydrides, acid halides, thioesters, phosphates, triphosphates, and β-sulfenyl derivatives.

The term “hydrocarbon” as used herein is a broad term, and is to be given its ordinary and customary meaning to a person of ordinary skill in the art (and is not to be limited to a special or customized meaning), and refers without limitation to any moiety comprising only carbon and hydrogen atoms. A functionalized or substituted hydrocarbon moiety has one or more substituents as described elsewhere herein.

The term “lipid” as used herein is a broad term, and is to be given its ordinary and customary meaning to a person of ordinary skill in the art (and is not to be limited to a special or customized meaning), and refers without limitation to saturated and unsaturated oils and waxes, derivatives, amides, glycerides, fatty acids, fatty alcohols, sterol and sterol derivatives, phospholipids, ceramides, sphingolipids, tocopherols, and carotenoids, among others.

The terms “pharmaceutically acceptable” as used herein is a broad term, and is to be given its ordinary and customary meaning to a person of ordinary skill in the art (and is not to be limited to a special or customized meaning), and refers without limitation to those compounds, materials, compositions, and/or dosage forms which are, within the scope of sound medical judgment, suitable for contact with the tissues of and/or for consumption by human beings and animals without excessive toxicity, irritation, allergic response, or other problem complications commensurate with a reasonable risk/benefit ratio.

The terms “pharmaceutically acceptable salts” and “a pharmaceutically acceptable salt thereof” as used herein are broad terms, and are to be given their ordinary and customary meaning to a person of ordinary skill in the art (and is not to be limited to a special or customized meaning), and refer without limitation to salts prepared from pharmaceutically acceptable, non-toxic acids or bases. Suitable pharmaceutically acceptable salts include metallic salts, e.g., salts of aluminum, zinc, alkali metal salts such as lithium, sodium, and potassium salts, alkaline earth metal salts such as calcium and magnesium salts; organic salts, e.g., salts of lysine, N,N′-dibenzylethylenediamine, chloroprocaine, choline, diethanolamine, ethylenediamine, meglumine (N-methylglucamine), procaine, and tris; salts of free acids and bases; inorganic salts, e.g., sulfate, hydrochloride, and hydrobromide; and other salts which are currently in widespread pharmaceutical use and are listed in sources well known to those of skill in the art, such as, for example, The Merck Index. Any suitable constituent can be selected to make a salt of the therapeutic agents discussed herein, provided that it is non-toxic and does not substantially interfere with the desired activity. In addition to salts, pharmaceutically acceptable precursors and derivatives of the compounds can be employed. Pharmaceutically acceptable amides, lower alkyl derivatives, and protected derivatives can also be suitable for use in compositions and methods of preferred embodiments. While it may be possible to administer the compounds of the preferred embodiments in the form of pharmaceutically acceptable salts, it is generally preferred to administer the compounds in neutral form.

The term “pharmaceutical composition” as used herein is a broad term, and is to be given its ordinary and customary meaning to a person of ordinary skill in the art (and is not to be limited to a special or customized meaning), and refers without limitation to a mixture of one or more compounds disclosed herein with other chemical components, such as diluents or carriers. The pharmaceutical composition facilitates administration of the compound to an organism. Pharmaceutical compositions can also be obtained by reacting compounds with inorganic or organic acids or bases. Pharmaceutical compositions will generally be tailored to the specific intended route of administration.

As used herein, a “carrier” as used herein is a broad term, and is to be given its ordinary and customary meaning to a person of ordinary skill in the art (and is not to be limited to a special or customized meaning), and refers without limitation to a compound that facilitates the incorporation of a compound into cells or tissues. For example, without limitation, dimethyl sulfoxide (DMSO) is a commonly utilized carrier that facilitates the uptake of many organic compounds into cells or tissues of a subject. Water, saline solution, ethanol, and mineral oil are also carriers employed in certain pharmaceutical compositions.

As used herein, a “diluent” as used herein is a broad term, and is to be given its ordinary and customary meaning to a person of ordinary skill in the art (and is not to be limited to a special or customized meaning), and refers without limitation to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, an “excipient” as used herein is a broad term, and is to be given its ordinary and customary meaning to a person of ordinary skill in the art (and is not to be limited to a special or customized meaning), and refers without limitation to a substance that is added to a pharmaceutical composition to provide, without limitation, bulk, consistency, stability, binding ability, lubrication, disintegrating ability etc., to the composition. A “diluent” is a type of excipient.

As used herein, a “subject” as used herein is a broad term, and is to be given its ordinary and customary meaning to a person of ordinary skill in the art (and is not to be limited to a special or customized meaning), and refers without limitation to an animal that is the object of treatment, observation or experiment. “Animal” includes cold- and warm-blooded vertebrates and invertebrates such as fish, shellfish, reptiles and, in particular, mammals. “Mammal” includes, without limitation, dolphins, mice, rats, rabbits, guinea pigs, dogs, cats, sheep, goats, cows, horses, primates, such as monkeys, chimpanzees, and apes, and, in particular, humans. In some embodiments, the subject is human.

As used herein, the terms “treating,” “treatment,” “therapeutic,” or “therapy” are broad terms, and are to be given their ordinary and customary meaning (and are not to be limited to a special or customized meaning) and, without limitation, do not necessarily mean total cure or abolition of the disease or condition. Any alleviation of any undesired markers, signs or symptoms of a disease or condition, to any extent, can be considered treatment and/or therapy. Furthermore, treatment may include acts that may worsen the patient's overall feeling of well-being or appearance.

The terms “therapeutically effective amount” and “effective amount” as used herein are broad terms, and are to be given its ordinary and customary meaning to a person of ordinary skill in the art (and are not to be limited to a special or customized meaning), and are used without limitation to indicate an amount of an active compound, or pharmaceutical agent, that elicits the biological or medicinal response indicated. For example, a therapeutically effective amount of compound can be the amount needed to prevent, alleviate or ameliorate markers or symptoms of a condition or prolong the survival of the subject being treated. This response may occur in a tissue, system, animal or human and includes alleviation of the signs or symptoms of the disease being treated. Determination of a therapeutically effective amount is well within the capability of those skilled in the art, in view of the disclosure provided herein. The therapeutically effective amount of the compounds disclosed herein required as a dose will depend on the route of administration, the type of animal, including human, being treated, and the physical characteristics of the specific animal under consideration. The dose can be tailored to achieve a desired effect, but will depend on such factors as weight, diet, concurrent medication and other factors which those skilled in the medical arts will recognize.

The term “solvents” as used herein is a broad term, and is to be given its ordinary and customary meaning to a person of ordinary skill in the art (and is not to be limited to a special or customized meaning), and refers without limitation to compounds with some characteristics of solvency for other compounds or means, that can be polar or nonpolar, linear or branched, cyclic or aliphatic, aromatic, naphthenic and that includes but is not limited to: alcohols, derivatives, diesters, ketones, acetates, terpenes, sulfoxides, glycols, paraffins, hydrocarbons, anhydrides, heterocyclics, among others.

Any percentages, ratios or other quantities referred to herein are on a weight basis, unless otherwise indicated.

### Odd Chain Fatty Acids

Fatty acids include saturated and unsaturated fatty acids as provided herein, fatty acids are referred to and described using conventional nomenclature as is employed by one of skill in the art. A saturated fatty acid includes no carbon-carbon double bonds. An unsaturated fatty acid includes at least one carbon-carbon double bond. A monounsaturated fatty acid includes only one carbon-carbon double bond. A polyunsaturated fatty acid includes two or more carbon-carbon double bonds. Double bonds in fatty acids are generally cis; however, trans double bonds are also possible. The position of double bonds can be indicated by Δn, where n indicates the lower numbered carbon of each pair of double-bonded carbon atoms. A shorthand notation in a form total #carbons: #double bonds, Δ_{double bond positions} can be employed. For example, 20:4Δ_{5,8,11,14} refers to a fatty acid having 20 carbon atoms and four double bonds, with the double bonds situated between the 5 and 6 carbon atom, the 8 and 9 carbon atom, the 11 and 12 carbon atom, and the 14 and 15 carbon atom, with carbon atom 1 being the carbon of the carboxylic acid group. Stearate (octadecanoate) is a saturated fatty acid. Oleate (cis-Δ9-octadecenoate) is a monounsaturated fatty acid, linolenate (all-cis-Δ9,12,15-octadecatrienoate) is a polyunsaturated fatty acid. The total number of carbons can be preceded by “C” and double bond positions can be unspecified, e.g., C20:4 referring to a fatty acid having 20 carbon atoms and four double bonds.

A fatty acid may be referred to by various names, for example, heptadecanoic acid may be referred to as heptadecylic acid, margaric acid, and n-heptadecylic acid, or C17:0. A fatty acid may be referred to by lipid numbers, as known in the art.

In some embodiments, the fatty acid can be an odd chain saturated fatty acid. In further embodiments, one or more fatty acids can include at least one odd chain saturated fatty acid.

Examples of odd chain fatty acids are margaric acid (heptadecanoic acid, C17:0), pelargonate (nonanoic acid, C9:0), undecanoic acid (C11:0), nonadecanoic acid (C19:0), pentadecanoic acid (C15:0), arachidonate ((5Z,8Z,11Z,14Z)-icosa-5,8,11,14-tetraenoic acid), adrenate (all-cis-7,10,13,16-docosatetraenoic acid), and osbond acid (all-cis-4,7,10,13,16-docosapentaenoic acid). Generally, the one or more odd chain fatty acids have from 9 carbon atoms to 31 carbon atoms (9, 11, 13, 15, 17, 19, 21, 23, 25, 27, 29, or 31 carbon atoms), for example, from 15 to 21 carbon atoms, for example 17 carbon atoms; however, in certain embodiments higher or lower odd numbers of carbon atoms can be acceptable. Generally, the one or more odd chain fatty acids are saturated; however, in certain embodiments mono or polyunsaturated odd chain fatty acids can be acceptable.

An odd chain fatty acid may include saturated or unsaturated hydrocarbon chains. An odd chain fatty acid may be present as a carboxylic derivative. An odd chain fatty acid may be present as a salt, for example, at the carboxylic group. In some embodiments, one odd chain fatty acid may be present, two odd chain fatty acids may be present, three odd chain fatty acids may be present, or more. In some embodiments, odd chain fatty acids in a mixture including a plurality of odd chain fatty acids may be distinguished by the amount of unsaturation, the length of the hydrocarbon chain, varying states of derivativeification, or by other structural features.

Odd chain fatty acids are found in trace amounts in some dairy products, including butter (see, e.g., Mansson H L (2008), Fatty acids in bovine milk fat, Food Nutr. Res. 52:4). Studies have demonstrated that increasing daily dietary intake of foods with odd chain fatty acids successfully increases serum or plasma levels (see, e.g., Benatar J. R., Stewart R. A. H. (2014), The effects of changing dairy intake on trans and saturated fatty acid levels-results from a randomized controlled study. Nutr. J. 13:32).

Generally, a fatty acid, such as an odd chain fatty acid can be provided as a free fatty acid, or a derivative thereof. Such derivatives include, but are not limited to, acyl glycerides. An acyl glyceride may be substituted with up to three acyl fatty acid esters. Thus, an acyl glyceride can be a monoacylglyceride (MAG), diacylglyceride (DAG), or a triacylglyceride (TAG). The glyceride can include more than one type of fatty acid ester. For example, a glyceride can include a heptdecanoate and a docosanoate. A glyceride can also be a structured triacylglyceride (STAG), a plasmalogen, or a phospholipid. The fatty acid ester can be in the sn1 position or the sn2 position, or both positions. The sn1 and sn2 positions can be substituted by the same or different fatty acid esters. As a non-limiting example, a structured triacylglyceride can be sn-1,3-C17-sn-2-oleoyl.

In some embodiments, a fatty acid can be provided as a free fatty acid, a cholesterol ester, a glycerol ester (including, but not limited to a monoacylglyceride (MAG), diacylglyceride (DAG), or a triacylglyceride (TAG)), a phospholipid (including, but not limited to, a phosphatidylcholine, a lysophosphatidylcholine, a phosphatidylethanolamine, a lysophosphatidylethanolamine, or a phosphatidylserine), a ceramide (including but not limited to a hexosyl ceramide) or a sphingolipid. A non-limiting example of a phophatidylcholine is 2,3-di-C17:0-phosphatidylcholine. A non-limiting example of a lysophophatidylcholine is 2-lyso-3-C17:0-phosphatidylcholine. In some embodiments, a derivative of a fatty acid can be a β-sulfenyl derivative. It is thought that β-sulfenyl derivative, such as an acid or ester, can be resistant to β-oxidation in the body. As a non-limiting example, the β-sulfenyl derivative of heptadecanoic acid is tetradecylthioacetic acid. Derivatives can be synthesized by standard methods known to those of skill in the art.

In some embodiments, a fatty acid may be provided as a constituent of a specific type of lipid, for example, a ceramide, a phospholipid, a sphingolipid, a membrane lipid, a glycolipid, or a triglyceride.

In some embodiments, a fatty acid, such as a very long even chain fatty acid, is provided in a bioavailable form. The term “bioavailability” refers to the fraction of an administered dose of unchanged drug that reaches the systemic circulation, one of the principal pharmacokinetic properties of drugs. By definition, when a medication is administered intravenously, its bioavailability is 100%. As employed herein, the term “bioavailable” refers to a form of the fatty acid that is successfully absorbed by the body when using methods of administration other than intravenous, for example, an oral therapeutic). In some embodiments, very long even chain fatty acid-based compositions may include adaptions that optimize absorption. In some embodiments, a very long even chain fatty acid can be provided as a structured triacylglyceride. In further embodiments, the fatty acid is in the sn-2 position of a structured triacylglyceride.

A pure or purified fatty acid may exist in various physical states. For example, heptadecanoic acid exists as an off-white powder that is stable at room temperature; this compound can be purchased in forms suitable for research purposes in small amounts from some commercial suppliers (for example, from Sigma-Aldrich corp., of St. Louis, MO). Other fatty acids, or salts or derivatives thereof, may exist as oils, solids, crystalline solids, or gases.

An odd chain fatty acid or the pharmaceutically acceptable salts or derivatives thereof, may be provided in a purity (e.g., a percentage of the fatty acid, or its pharmaceutically acceptable salts or derivatives, in a bulk form) of at least about 10%, at least about 20%, at least about 30%, at least about 40%, at least about 50%, at least about 60%, at least about 70%, at least about 80%, at least about 90%, at least about 95%, at least about 98%, at least about 99%, at least about 99.9%, at least about 99.99%, or substantially pure, wherein substantially pure may include, but not be limited to, a product with impurities at a level such that no physiological effect from the presence of the impurities is detectable. A mixture of fatty acids, such as, for example, odd chain fatty acids and/or very long even chain fatty acids, or pharmaceutically acceptable salts or derivatives thereof, may be present in a purity of at least about 10%, at least about 20%, at least about 30%, at least about 40%, at least about 50%, at least about 60%, at least about 70%, at least about 80%, at least about 90%, at least about 95%, at least about 98%, at least about 99%, at least about 99.9%, at least about 99.99%, or substantially pure. The fatty acid, or a mixture thereof, or a pharmaceutically acceptable salt or derivative thereof, may be free from other fatty acids or fatty acid derivatives, may be free from triglycerides, or may be free from phospholipids. Without limitation, an odd chain fatty acid as provided herein may be substantially free from even chain fatty acids, singly or taken as a group; even chain fatty acids include, for example, myristic acid (C14:0), palmitic acid (C16:0), or stearic acid (C18:0). In some embodiments, an odd chain fatty acid as provided herein may be substantially free from short-chain fatty acids (SCFA, e.g., a fatty acid with 2-6 carbon atoms), medium-chain fatty acids (MCFA, e.g., a fatty acid with 7-12 carbon atoms), long-chain fatty acids (LCFA, e.g., a fatty acid with 13-22 carbon atoms), or very long chain fatty acids (VLCFA, e.g., a fatty acid with 23 or more carbon atoms).

A fatty acid, such as an odd chain fatty acid or a pharmaceutically acceptable salt or derivative thereof, may be from any source. In some embodiments, a fatty acid, or its pharmaceutically acceptable salts or derivatives, may be present in natural sources, may be isolated from natural sources, may be semi-synthetic, may be synthetic, or may be a mixture of one or more of these. The fatty acid, or its pharmaceutically acceptable salts or derivatives, may be produced in a laboratory, may be produced in nature, may be produced by enzymatic processes, may be produced by wild microbes, may be produced by genetically modified microbes, may be isolated from animal tissues, may be produced by chemical synthesis, or may be produced by a plurality of these processes.

The fatty acid may be derived from natural sources, e.g., fish oils, or can be synthesized by methods as are known in the art. In some embodiments, the fatty acid may be contaminated with undesired components present in unrefined or unpurified natural products. In such situations, it can be desirable to remove undesired components, or to increase the concentration of desired components using known separation or purification techniques.

In any compound described, all tautomeric forms are also intended to be included. Without limitation, all tautomers of carboxylic groups are intended to be included.

In any compound described herein having one or more double bond(s) generating geometrical isomers that can be defined as E or Z, each double bond may independently be E or Z, or a mixture thereof.

Where compounds disclosed herein have unfilled valencies, then the valencies are to be filled with hydrogens or isotopes thereof, e.g., hydrogen-1 (protium) and hydrogen-2 (deuterium).

The fatty acid, such as an odd chain fatty acid, as described herein, includes crystalline forms (also known as polymorphs, which include the different crystal packing arrangements of the same elemental composition of a compound), amorphous phases, salts, solvates, and hydrates. In some embodiments, the compounds described herein exist in solvated forms with pharmaceutically acceptable solvents such as water, ethanol, or the like. In other embodiments, the compounds described herein exist in unsolvated form. Solvates contain either stoichiometric or non-stoichiometric amounts of a solvent, and may be formed during the process of crystallization with pharmaceutically acceptable solvents such as water, ethanol, or the like. Hydrates are formed when the solvent is water, or alcoholates are formed when the solvent is alcohol. In addition, the compounds provided herein can exist in unsolvated as well as solvated forms. In general, the solvated forms are considered equivalent to the unsolvated forms for the purposes of the compounds and methods provided herein.

The compounds described herein can be labeled isotopically. In some circumstances, substitution with isotopes such as deuterium may afford certain therapeutic advantages resulting from greater metabolic stability, such as, for example, increased in vivo half-life or reduced dosage requirements. Isotopic substitution may be beneficial in monitoring subject response to administration of a compound, for example, by providing opportunity for monitoring of the fate of an atom in a compound. Each chemical element as represented in a compound structure may include any isotope of said element. For example, in a compound structure a hydrogen atom may be explicitly disclosed or understood to be present in the compound. At any position of the compound that a hydrogen atom may be present, the hydrogen atom can be any isotope of hydrogen, including but not limited to hydrogen-1 (protium) and hydrogen-2 (deuterium). Thus, reference herein to a compound encompasses all potential isotopic forms unless the context clearly dictates otherwise.

The prevalence of various fatty acids in the diet has been correlated to the occurrence of metabolic syndrome in subjects (see, e.g., Forouhi N, Koulman A, Sharp S, Imamura F, Kröger J, Schulze M, et al. (2014), Differences in the prospective association between individual plasma phospholipid saturated fatty acids and incident type 2 diabetes: the EPIC-InterAct case-cohort study. Lancet Diabetes Endocrinol. 2:810-8). Indeed, whole-fat dairy consumption has been correlated with a decreased risk of metabolic syndrome markers (see, e.g., Kratz M, Marcovina S, Nelson J E, Yeh M M, Kowdley K V, Callahan H S, et al. (2014), Dairy fat intake is associated with glucose tolerance, hepatic and systemic insulin sensitivity, and liver fat but not beta-cell function in humans, Am. J. Clin. Nutr., 99:1385-96).

The mechanism(s) by which odd chain saturated fatty acid(s) have a beneficial effect are not well understood. Without wishing to be limited by theory, it is thought that fatty acids, or derivatives thereof, can be elongated (increased in chain length) or chain shortened by metabolic processes in the body, to form different fatty acids, or derivatives thereof. Peroxidation of certain fatty acids may create products with signaling characteristics in the body. It is thought that fatty acids of certain chain length create signaling products that substantially contribute to one or more conditions provided herein. In some embodiments, an odd chain fatty acid is elongated to form a very long chain fatty acid, such as a very long even chain fatty acid. In further embodiments, a very long even chain fatty acid can be chain-shortened to an odd chain fatty acid. Levels of very long even chain fatty acids in the body may increase following administration of one or more odd chain fatty acids. Levels of odd chain fatty acids in the body may increase following administration of one or more very long even chain fatty acids.

### Pharmaceutical Compositions Including One or More Fatty Acids

Formulations including a fatty acid, such as an odd chain fatty acid or a very long even chain fatty acid, or a salt or derivative thereof, and at least one excipient are provided. It is generally preferred to administer the compounds of the embodiments in oral formulations; however, other routes of administration are also contemplated.

The pharmaceutical compositions described herein can be administered by themselves to a subject, or in compositions where they are mixed with other active agents, as in combination therapy, or with carriers, diluents, excipients or combinations thereof. Formulation is dependent upon the route of administration chosen. Techniques for formulation and administration of the compounds described herein are known to those skilled in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

The pharmaceutical compositions disclosed herein may be manufactured by a process that is itself known, e.g., by means of conventional mixing, dissolving, granulating, dragee-making, levigating, emulsifying, encapsulating, entrapping, tableting, or extracting processes. Many of the compounds used in the pharmaceutical combinations disclosed herein may be provided as salts with pharmaceutically acceptable counterions.

Multiple techniques of administering a compound exist in the art including, but not limited to, oral, rectal, topical, aerosol, injection and parenteral delivery, including intramuscular, subcutaneous, intravenous, intramedullary injections, intrathecal, direct intraventricular, intraperitoneal, intranasal and intraocular injections. Contemplated herein is any combination of the forgoing, or other methods as would be known to one of ordinary skill in the art (see, e.g., “Remington: The Science and Practice of Pharmacy”, Lippincott Williams & Wilkins; 20th edition (Jun. 1, 2003) and “Remington's Pharmaceutical Sciences,” Mack Pub. Co.; 18th and 19th editions (December 1985, and June 1990, respectively).

In practice, a fatty acid, such as an odd chain saturated fatty acid or a salt or derivative thereof, may be combined as the active ingredient in intimate admixture with a pharmaceutical carrier according to conventional pharmaceutical compounding techniques. The carrier can take a wide variety of forms depending on the form of preparation desired for administration. Thus, the pharmaceutical compositions provided herein can be presented as discrete units suitable for oral administration such as capsules, cachets or tablets each containing a predetermined amount of the active ingredient. Further, the compositions can be presented as an oil, a powder, as granules, as a solution, as a suspension in an aqueous liquid, as a non-aqueous liquid, as an oil-in-water emulsion, or as a water-in-oil liquid emulsion. In addition to the common dosage forms set out above, the compounds provided herein, or pharmaceutically acceptable salts or derivatives thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

A formulation may also be administered in a local rather than systemic manner, for example, via injection of the compound directly into the infected area, often in a depot or sustained release formulation. Furthermore, a targeted drug delivery system might be used, for example, in a liposome coated with a tissue specific antibody.

The pharmaceutical compositions may contain a fatty acid, such as an odd chain fatty acid, or a salt or derivative thereof, in an amount effective for the desired therapeutic effect. In some embodiments, the pharmaceutical compositions are in a unit dosage form and comprise from about 0.1 mg or less to about 5000 mg or more per unit dosage form. In further embodiments, the pharmaceutical compositions comprise from about 1 to about 500 mg per unit dosage form or from about 500 to 5000 mg per unit dosage form. Such dosage forms may be solid, semisolid, liquid, an emulsion, or adapted for delivery via aerosol or the like for inhalation administration.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, lower alcohols, and water. Examples of gaseous carriers include carbon dioxide and nitrogen.

Pharmaceutical compositions provided herein can be prepared as solutions or suspensions of the active compound(s) in water. A suitable surfactant can be included such as, for example, hydroxypropylcellulose. Dispersions can also be prepared in glycerol, liquid polyethylene glycols, and mixtures thereof in oils. Further, a preservative can be included to, for example, prevent the detrimental growth of microorganisms.

Pharmaceutical compositions provided herein suitable for injectable use include sterile aqueous solutions or dispersions. Furthermore, the compositions can be in the form of sterile powders for the extemporaneous preparation of such sterile injectable solutions or dispersions. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound provided herein, or pharmaceutically acceptable salt or derivative thereof, can also be prepared in powder or liquid concentrate form for dilution.

The fatty acid, such as an odd chain saturated fatty acid, or a salt or derivative thereof, can be formulated as a liposome. The fatty acid can be a component of the lipid portion of the liposome or can be encapsulated in the aqueous portion of the liposome. The fatty acid, such as an odd chain fatty acid, or a salt or derivative thereof, can also be coformulated with a cyclodextrin. The cyclodextrin can be, for example, hydroxypropyl-β-cyclodextrin or a sulfobutylether cyclodextrin.

Contemplated herein are compositions including a fatty acid, such as an odd chain saturated fatty acid, or a salt or derivative thereof in combination with at least one additional active agent. A fatty acid, such as an odd chain saturated fatty acid, or a salt or derivative thereof, and the at least one additional active agent(s) may be present in a single formulation or in multiple formulations provided together, or may be unformulated (for example, free of excipients and carriers). In some embodiments, a fatty acid, such as an odd chain saturated fatty acid, or a salt or derivative thereof, can be administered with one or more additional agents together in a single composition. For example, a compound of a fatty acid, such as an odd chain saturated fatty acid, or a salt or derivative thereof, can be administered in one composition, and at least one of the additional agents can be administered in a second composition. In a further embodiment, a fatty acid, such as an odd chain saturated fatty acid, or a salt or derivative thereof and the at least one additional active agent(s) are co-packaged in a kit. For example, a drug manufacturer, a drug reseller, a physician, a compounding shop, or a pharmacist can provide a kit comprising a disclosed compound or product and another component for delivery to a patient.

Some embodiments described herein relate to a pharmaceutical composition, which can include a therapeutically effective amount of one or more compounds described herein (e.g., a fatty acid, such as an odd chain saturated fatty acid or a pharmaceutically acceptable salt or derivative thereof) and a pharmaceutically acceptable carrier, diluent, excipient or combination thereof. The pharmaceutical composition can include a fatty acid such as an odd chain saturated fatty acid, or a salt or derivative thereof in, for example, >1%, ≥2%, ≥3%, ≥4%, ≥5%, ≥6%, ≥7%, ≥8%, ≥9%, ≥10%, ≥20%, ≥30%, ≥40%, ≥50%, ≥60%, ≥70%, ≥80%, ≥90%, ≥95%, or ≥98% of the composition. In some embodiments, the pharmaceutical composition can include a plurality of fatty acids, such as one or more of an odd chain saturated fatty acid and/or a very long even chain fatty acid, or salts or derivatives thereof in, for example, >1%, ≥2%, ≥3%, ≥4%, ≥5%, ≥6%, ≥7%, ≥8%, ≥9%, ≥10%, ≥20%, ≥30%, ≥40%, ≥50%, ≥60%, ≥70%, ≥80%, ≥90%, ≥95%, or ≥98% of the composition.

### Foodstuffs

Foodstuffs and other comestibles including a fatty acid, such as an odd chain saturated fatty acid, or a salt or derivative thereof, are provided, wherein an amount of the fatty acid in the foodstuff has been fortified (e.g., enriched or concentrated). A fatty acid, such as an odd chain saturated fatty acid, provided herein may be added to foodstuffs for consumption by a subject. The fatty acid, such as an odd chain saturated fatty acid, may be integrated into one or more ingredients of a foodstuff. The fatty acid, such as an odd chain saturated fatty acid, may be prepared as an ingredient, or may be unprepared. The compound, or preparation including the compound, may be added prior to preparation, during preparation, or following preparation. Preparation may without limitation include cooking, mixing, flavoring, seasoning, blending, boiling, frying, baking, or other processes known in the art. Fortification is preferably at a level so as to provide a therapeutic daily dosage of the fatty acid as described elsewhere herein; however, beneficial effects may also be obtained at amounts below such dosages.

A fatty acid, such as an odd chain saturated fatty acid, or salt or derivative thereof, as provided herein may be present as a constituency in foodstuffs by operation of processes known in nature, for example, by altering the metabolic processes of a plant, animal, bacteria, or fungus. Genetic alteration of a plant, animal, bacteria, or fungus to increase the concentration of a fatty acid, such as an odd chain saturated fatty acid, or a salt or derivative thereof, is contemplated. By way of example, the fatty acid can be present in the foodstuff in a concentration of at least about 1%, at least about 2%, at least about 3%, at least about 4%, at least about 5%, at least about 6%, at least about 7%, at least about 8%, at least about 9%, at least about 10%, at least about 20%, at least about 30%, at least about 40%, at least about 50%, or higher, for example, 1% to 2% or 3% or 4% or 5% or 6% or 7% or 8% or 9% or 10% or 20% or 30% or 40% or 50%.

### Indications

Provided are compositions and methods for treating aging-associated conditions. These conditions include but are not limited to hypercholesterolemia, obesity, thrombosis, fibrosis, wound healing, hyperglobulinemia, hypersensitivity, and cancer; dyslipidemia (including elevated total cholesterol or elevated LDL-cholesterol levels), thrombosis (including venous thrombosis, deep vein thrombosis, Paget-Schroetter disease, Budd-Chiari syndrome, portal vein thrombosis, renal vein thrombosis, cerebral venous sinus thrombosis, jugular vein thrombosis, cavernous sinus thrombosis, arterial thrombosis, stroke, myocardial infarctions, limb ischemia, and hepatic artery thrombosis), fibrotic diseases (including nonalcoholic steatohepatitis (NASH), pulmonary fibrosis, cystic fibrosis, idiopathic pulmonary fibrosis, radiation-induced lung injury, liver fibrosis, cirrhosis, biliary atresia, cardiac fibrosis, atrial fibrosis, endomyocardial fibrosis, old myocardial infarction, brain fibrosis, glial scars, arterial stiffness, arthrofibrosis, Crohn's disease, Dupuytren's contracture, keloid, epidermal and dermal scarring, mediastinal fibrosis, myelofibrosis, Peyronie's disease, nephrogenic systemic fibrosis, progressive massive fibrosis, pneumnoconiosis, retroperitoneal fibrosis, sclerodoma, systemic sclerosis, and adhesive capsulitis), wound healing and skin repair (including delayed wound healing due to age, obesity, chronic diseases, immunosuppression, nutritional status, or vascular insufficiency), hyperglobulinemia and associated conditions, hypersensitivity (including allergies and autoimmune diseases, autoimmune hemolytic anemia, rheumatic heart disease, thrombocytopenia, eryrthroblastosis fetalis, Goodpature's syndrome, Graves' disease, myasthenia gravis, serum sickness, arthus reaction, post streptococcal glomerulonephritis, membranous nephropathy, reactive arthritis, lupus nephritis, systemic lupus erythematosus, extrinsic allergic alveolitis, hypersensitivity pneumonitis, rheumatoid arthritis, multiple sclerosis, Graves' disease, or myasthenia gravis), and neoplastic diseases including cancer (such as, for example, lymphoma, Hodgkin disease and hepatocellular carcinoma).

Aging refers to a series of morphological and functional changes in an organism which take place over time. The term also refers to the deterioration of the biological functions after an organism has attained its maximum reproductive potential. It is thought that inflammation is a condition associated with aging through mutation to mitochondrial DNA and other processes.

In some embodiments, the compositions and methods provided herein are indicated for treatment, prophylaxis, prevention or maintenance of aging-associated conditions, including hypercholesterolemia, obesity, thrombosis, fibrosis, wound healing, hyperglobulinemia, hypersensitivity, or cancer.

Without wishing to be limited by theory, it is thought that increasing odd chain saturated fatty acid free fatty acid or phospholipid levels in the serum, plasma, and cells to targeted concentrations may decrease aging-associated conditions.

In some embodiments, the methods provided herein increase levels of serum, plasma, or erythrocyte membrane odd chain fatty acids.

In some embodiments, levels of serum, plasma, or erythrocyte membrane very long even chain fatty acids may increase following administration of one or more odd chain fatty acids, or a salt or derivative thereof.

In some embodiments, the condition treated is anemia of chronic disease.

In some embodiments, the condition treated is autoimmune disease.

In some embodiments, the compositions and methods provided herein modulate a marker of a condition associated with aging. In certain embodiments, the marker is serum, plasma, or red blood cell membrane odd chain fatty acid percentage; serum, plasma, or red blood cell membrane concentration of an odd chain fatty acid; serum plasma, or red blood cell membrane total odd chain fatty acid; erythrocyte sedimentation rate, alkaline phosphatase, serum ferritin, CRP (C reactive protein), IL-6 and TNFα (and other cytokines associated with insulin resistance), c-Jun N-terminal kinase (JNK), ATM (Ataxia Telangiectasia Mutated) or monocyte-chemoattractant protein-1. In some embodiments, the odd chain fatty acid is measured as a constituent of glycolipids. In further embodiments, the odd chain fatty acid is measured as a constituent of phospholipids. In still further embodiments, the marker is serum or red blood cell membrane very long even chain fatty acid percentage, serum concentration of a very long even chain fatty acid, serum total very long even chain fatty acids.

In some embodiments, the methods provided herein include the step of measuring the concentration of a marker of a condition associated with aging. One of skill in the art will be able to perform suitable methods for such measurements, including but not limited to those described herein.

Provided herein are methods for treating including the step of administering a dose of a fatty acid, such as an odd chain fatty acid or a very long even chain fatty acid, at a predetermined interval, or at an interval left to the discretion of the subject.

In some embodiments, the compounds and methods provided herein may provide a threshold serum, plasma, or red blood cell membrane percentage of an odd chain fatty acid relative to all serum, plasma, or red blood cell membrane fatty acids, respectively. For example, the threshold value may be a value of about 0.05% or lower to 90% or higher, e.g., a value of at least about 0.05%, at least about 0.1%, at least about 0.2%, at least about 0.3%, at least about 0.4%, at least about 0.5%, at least about 0.6%, at least about 0.7%, at least about 0.8%, at least about 0.9%, at least about 1.0%, at least about 1.1%, at least about 1.2%, at least about 1.3%, at least about 1.4%, at least about 1.5%, at least about 1.6%, at least about 1.7%, at least about 1.8%, at least about 1.9%, at least about 2.1%, at least about 2.2%, at least about 2.3%, at least about 2.4%, at least about 2.5%, at least about 2.6%, at least about 2.7%, at least about 2.8%, at least about 2.9%, at least about 3.0%, at least about 3.5%, at least about 4.0%, at least about 4.5%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or more than 90%.

In some embodiments, the compounds and methods provided herein may provide an increase above a baseline value (e.g., pretreatment value in a patient being treated, or general value observed in a particular patient population) in a serum or plasma concentration of an odd chain fatty acid, or red blood cell membrane concentration of an odd chain fatty acid. For example, a serum or plasma odd chain fatty acid or red blood cell membrane concentration of an odd chain fatty acid may be increased by at least about 1 μg/ml, at least about 2 μg/ml, at least about 3 μg/ml, at least about 4 μg/ml, at least about 5 μg/ml, at least about 6 μg/ml, at least about 7 μg/ml, at least about 8 μg/ml, at least about 9 μg/ml, at least about 10 μg/ml, at least about 15 μg/ml, at least about 20 μg/ml, at least about 25 μg/ml, at least about 30 μg/ml, at least about 35 μg/ml, at least about 40 μg/ml, at least about 45 μg/ml, at least about 50 μg/ml, or more than 50 μg/ml. In some embodiments, the serum concentration of an odd chain fatty acid, or red blood cell membrane concentration of an odd chain fatty acid may increase above a baseline value (e.g., pretreatment value in a patient being treated, or general value observed in a particular patient population) by at least about 0.01×10⁻⁴ M, at least about 0.05×10⁻⁴ M, at least about 0.1×10⁻⁴ M, at least about 0.2×10⁻⁴ M, at least about 0.3×10⁻⁴ M, at least about 0.4×10⁻⁴ M, at least about 0.5×10⁻⁴ M, at least about 0.6×10⁻⁴ M, at least about 0.7×10⁻⁴ M, at least about 0.8×10⁻⁴ M, at least about 0.9×10⁻⁴ M, at least about 1×10⁻⁴ M, at least about 2×10⁻⁴ M, or at least about 3×10⁻⁴ M.

In some embodiments, the compounds and methods provided herein may provide an increase in serum or plasma total odd chain fatty acids, or red blood cell membrane total odd chain fatty acids. For example, serum total odd chain fatty acids, or red blood cell membrane total odd chain fatty acids, may be increased above a baseline value (e.g., pretreatment value in a patient being treated, or general value observed in a particular patient population) by at least about 5 μg/ml, at least about 6 μg/ml, at least about 7 μg/ml, at least about 8 μg/ml, at least about 9 μg/ml, at least about 10 μg/ml, at least about 15 μg/ml, at least about 20 μg/ml, at least about 25 μg/ml, at least about 30 μg/ml, at least about 35 μg/ml, at least about 40 μg/ml, at least about 45 μg/ml, at least about 50 μg/ml, at least about 60 μg/ml, at least about 70 μg/ml, at least about 80 μg/ml, at least about 90 μg/ml, at least about 100 μg/ml, at least about 150 μg/ml, at least about 200 μg/ml, at least about 250 μg/ml, at least about 300 μg/ml, at least about 350 μg/ml, at least about 400 μg/ml, at least about 450 μg/ml, at least about 500 μg/ml, or more than 500 μg/ml.

In some embodiments, the compounds and methods provided herein may provide an increase above a baseline value (e.g., pretreatment value in a patient being treated, or general value observed in a particular patient population) in a serum, plasma, or red blood cell membrane odd chain fatty acids relative to all serum or red blood cell membrane fatty acids, respectively. For example, a serum, plasma, or red blood cell membrane odd chain fatty acid may be increased above a baseline value (e.g., pretreatment value in a patient being treated, or general value observed in a particular patient population) by at least about 0.01%, at least about 0.05%, at least about 0.1%, at least about 0.2%, at least about 0.3%, at least about 0.4%, at least about 0.5%, at least about 0.6%, at least about 0.7%, at least about 0.8%, at least about 0.9%, at least about 1%, at least about 1.1%, at least about 1.2%, at least about 1.3%, at least about 1.4%, at least about 1.5%, at least about 1.6%, at least about 1.7%, at least about 1.8%, at least about 1.9%, at least about 2%, at least about 2.1%, at least about 2.2%, at least about 2.3%, at least about 2.4%, at least about 2.5%, at least about 2.6%, at least about 2.7%, at least about 2.8%, at least about 2.9%, at least about 3%, at least about 3.5%, at least about 4%, at least about 4.5%, at least about 5%, or more than 5%.

In some embodiments, the compounds and methods provided herein may provide a reduction in elevated erythrocyte sedimentation rate.

In some embodiments, the compounds and methods provided herein may provide a reduction in elevated alkaline phosphatase.

In some embodiments, the compounds and methods provided herein may provide a reduction in serum ferritin. For example, serum ferritin may be reduced below a baseline value (e.g., pretreatment value in a patient being treated, or general value observed in a particular patient population) by at least about 10 ng/ml, at least about 100 ng/ml, at least about 200 ng/ml, at least about 300 ng/ml, at least about 400 ng/ml, at least about 500 ng/ml, at least about 600 ng/ml, at least about 700 ng/ml, at least about 800 ng/ml, at least about 900 ng/ml, at least about 1000 ng/ml, at least about 1100 ng/ml, at least about 1200 ng/ml, at least about 1300 ng/ml, at least about 1400 ng/ml, at least about 1500 ng/ml, at least about 2000 ng/ml, at least about 2500 ng/ml, at least about 3000 ng/ml, at least about 3500 ng/ml, at least about 4000 ng/ml, at least about 4500 ng/ml, at least about 5000 ng/ml, at least about 6000 ng/ml, at least about 7000 ng/ml, at least about 8000 ng/ml, at least about 9000 ng/ml, at least about 10000 ng/ml, or more than 10000 ng/ml.

In some embodiments, the compounds and methods provided herein may provide a reduction in serum ferritin below a specified level. For example, serum ferritin may be reduced below about 20000 ng/ml, about 15000 ng/ml, about 12000 ng/ml, about 10000 ng/ml, about 8000 ng/ml, about 5000 ng/ml, about 2000 ng/ml, about 1000 ng/ml, or about 500 ng

In some embodiments, an odd chain fatty acid (e.g., a saturated odd chain fatty acid) is administered to maintain serum or plasma total percent of the odd chain fatty acid, or all odd chain fatty acids, above a predetermined threshold value. In variations of these embodiments, the odd chain fatty acid is heptadecanoic acid. In further variations, the odd chain fatty acid is administered to maintain serum phospholipid percent of the odd chain fatty acid, or all odd chain fatty acids, above about 0.1%, about 0.2%, about 0.3%, about 0.4%, about 0.5%, about 0.6%, about 0.7%, about 0.8%, about 0.9%, about 1%, about 1.2%, about 1.4%, about 1.6%, about 1.8%, about 2%, about 2.2%, about 2.4%, or about 2.6%.

In some embodiments, the compounds and methods provided herein may provide a threshold serum, plasma, or red blood cell membrane percentage of a very long even chain fatty acid relative to all serum or red blood cell membrane fatty acids, respectively. For example, the threshold value may be a value of about 0.05% or lower to 90% or higher, e.g., a value of at least about 0.05%, at least about 0.1%, at least about 0.2%, at least about 0.3%, at least about 0.4%, at least about 0.5%, at least about 0.6%, at least about 0.7%, at least about 0.8%, at least about 0.9%, at least about 1.0%, at least about 1.1%, at least about 1.2%, at least about 1.3%, at least about 1.4%, at least about 1.5%, at least about 1.6%, at least about 1.7%, at least about 1.8%, at least about 1.9%, at least about 2.1%, at least about 2.2%, at least about 2.3%, at least about 2.4%, at least about 2.5%, at least about 2.6%, at least about 2.7%, at least about 2.8%, at least about 2.9%, at least about 3.0%, at least about 3.5%, at least about 4.0%, at least about 4.5%, at least about 5%, at least about 6%, at least about 7%, at least about 8%, at least about 9%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90%, or more than 90%.

In some embodiments, the compounds and methods provided herein may provide an increase above a baseline value (e.g., pretreatment value in a patient being treated, or general value observed in a particular patient population) in a serum or plasma concentration of a very long even chain fatty acid, or red blood cell membrane concentration of a very long even chain fatty acid. For example, a serum very long even chain fatty acid or red blood cell membrane concentration of a very long even chain fatty acid may be increased by at least about 0.01 μg/ml, at least about 0.05 μg/ml, at least about 0.1 μg/ml, at least about 0.4 μg/ml, 1 μg/ml, at least about 2 μg/ml, at least about 3 μg/ml, at least about 4 μg/ml, at least about 5 μg/ml, at least about 6 μg/ml, at least about 7 μg/ml, at least about 8 μg/ml, at least about 9 μg/ml, at least about 10 μg/ml, at least about 15 μg/ml, at least about 20 μg/ml, at least about 25 μg/ml, at least about 30 μg/ml, at least about 35 μg/ml, at least about 40 μg/ml, at least about 45 μg/ml, at least about 50 μg/ml, or more than 50 μg/ml. In some embodiments, the serum concentration of a very long even chain fatty acid, or red blood cell membrane concentration of a very long even chain fatty acid may increase above a baseline value (e.g., pretreatment value in a patient being treated, or general value observed in a particular patient population) by at least about 0.001×10⁻⁴ M, at least about 0.005×10⁻⁴ M, at least about 0.05×10⁻⁴ M, at least about 0.01×10⁻⁴ M, at least about 0.05×10⁻⁴ M, at least about 0.1×10⁻⁴ M, at least about 0.2×10⁻⁴ M, at least about 0.3×10⁻⁴ M, at least about 0.4×10⁻⁴ M, at least about 0.5×10⁻⁴ M, at least about 0.6×10⁻⁴ M, at least about 0.7×10⁻⁴ M, at least about 0.8×10⁻⁴ M, at least about 0.9×10⁻⁴ M, at least about 1×10⁻⁴ M, at least about 2×10⁻⁴ M, or at least about 3×10⁻⁴ M.

In some embodiments, the compounds and methods provided herein may provide an increase in serum or plasma total very long even chain fatty acids, or red blood cell membrane total very long even chain fatty acids. For example, serum total very long even chain fatty acids, or red blood cell membrane total very long even chain fatty acids, may be increased above a baseline value (e.g., pretreatment value in a patient being treated, or general value observed in a particular patient population) by at least about 0.05 μg/ml, at least about 0.1 μg/ml, at least about 0.5 μg/ml, at least about 1 μg/ml, at least about 5 μg/ml, at least about 6 μg/ml, at least about 7 μg/ml, at least about 8 μg/ml, at least about 9 μg/ml, at least about 10 μg/ml, at least about 15 μg/ml, at least about 20 μg/ml, at least about 25 μg/ml, at least about 30 μg/ml, at least about 35 μg/ml, at least about 40 μg/ml, at least about 45 μg/ml, at least about 50 μg/ml, at least about 60 μg/ml, at least about 70 μg/ml, at least about 80 μg/ml, at least about 90 μg/ml, at least about 100 μg/ml, at least about 150 μg/ml, at least about 200 μg/ml, at least about 250 μg/ml, at least about 300 μg/ml, at least about 350 μg/ml, at least about 400 μg/ml, at least about 450 μg/ml, at least about 500 μg/ml, or more than 500 μg/ml.

In some embodiments, a composition or method provided herein may provide an increase in red blood cell count. For example, a red blood cell count level may be increased above a baseline value (e.g., pretreatment value in a patient being treated, or general value observed in a particular patient population) by at least about 0.1 cells/μL, at least about 0.2 cells/μL, at least about 0.3 cells/μL, at least about 0.4 cells/μL, at least about 0.5 cells/μL, at least about 0.6 cells/μL, at least about 0.7 cells/μL, at least about 0.8 cells/μL, at least about 0.9 cells/μL, at least about 1 cell/μL, at least about 1.2 cells/μL, at least about 1.4 cells/μL, at least about 1.6 cells/μL, or at least about 2 cells/μL.

### Combination Therapies

In some embodiments, the compounds disclosed herein, such as an odd chain fatty acid, or a salt or derivative thereof, or a very long even chain fatty acid, or a salt or derivative thereof, or a pharmaceutical composition that includes a compound described herein, or a salt or derivative thereof, may be used in combination with one or more additional active agents. Examples of additional active agents that can be used in combination with a compound of an odd chain fatty acid, or a salt or derivative thereof, or a composition that includes a compound of an odd chain fatty acid, or a salt or derivative thereof, include, but are not limited to, agents currently used for treating conditions provided herein, and as otherwise known to medical science.

In some embodiments, a compound of an odd chain fatty acid, or a salt or derivative thereof, or a composition that includes a compound of an odd chain fatty acid, or a salt or derivative thereof, can be used with one, two, three or more additional active agents described herein. Such agents include, but are not limited to, a second fatty acid, such as an odd chain fatty acid or a very long even chain fatty acid, or a salt or derivative thereof. In some embodiments, a composition can include at least one odd chain fatty acid, or a salt or derivative thereof, and at least one very long even chain fatty acid, or a salt or derivative thereof.

In some embodiments, a compound of an odd chain fatty acid, or a salt or derivative thereof, or a composition that includes a compound of an odd chain fatty acid, or a salt or derivative thereof, can be used (for example, administered or ingested) in combination with another agent or agents for treatment, prevention, maintenance, or prophylaxis of a condition provided herein including aging-associated conditions, including hypercholesterolemia, obesity, thrombosis, fibrosis, wound healing, hyperglobulinemia, hypersensitivity, and cancer cell proliferation or for modulation of markers of the condition. In some embodiments, the condition can be dyslipidemia (including elevated total cholesterol or elevated LDL-cholesterol levels), thrombosis (including venous thrombosis, deep vein thrombosis, Paget-Schroetter disease, Budd-Chiari syndrome, portal vein thrombosis, renal vein thrombosis, cerebral venous sinus thrombosis, jugular vein thrombosis, cavernous sinus thrombosis, arterial thrombosis, stroke, myocardial infarctions, limb ischemia, and hepatic artery thrombosis), fibrotic diseases (including nonalcoholic steatohepatitis (NASH), pulmonary fibrosis, cystic fibrosis, idiopathic pulmonary fibrosis, radiation-induced lung injury, liver fibrosis, cirrhosis, biliary atresia, cardiac fibrosis, atrial fibrosis, endomyocardial fibrosis, old myocardial infarction, brain fibrosis, glial scars, arterial stiffness, arthrofibrosis, Crohn's disease, Dupuytren's contracture, keloid, epidermal and dermal scarring, mediastinal fibrosis, myelofibrosis, Peyronie's disease, nephrogenic systemic fibrosis, progressive massive fibrosis, pneumnoconiosis, retroperitoneal fibrosis, sclerodoma, systemic sclerosis, and adhesive capsulitis), wound healing and skin repair (including delayed wound healing due to age, obesity, chronic diseases, immunosuppression, nutritional status, or vascular insufficiency), hyperglobulinemia and associated conditions, hypersensitivity (including allergies and autoimmune diseases, autoimmune hemolytic anemia, rheumatic heart disease, thrombocytopenia, eryrthroblastosis fetalis, Goodpature's syndrome, Graves' disease, myasthenia gravis, serum sickness, arthus reaction, post streptococcal glomerulonephritis, membranous nephropathy, reactive arthritis, lupus nephritis, systemic lupus erythematosus, extrinsic allergic alveolitis, hypersensitivity pneumonitis, rheumatoid arthritis, multiple sclerosis, Graves' disease, or myasthenia gravis), and neoplastic diseases including cancer (such as, for example, lymphoma, Hodgkin disease and hepatocellular carcinoma).

For example, a compound of a fatty acid, such as an odd chain fatty acid, disclosed herein can be used in combination with one or more agents selected from statins (e.g., atorvastatin (Lipitor), fluvastatin (Lescol), lovastatin (Altoprev), pitavastatin (Livalo), pravastatin (Pravachol), rosuvastatin (Crestor), simvastatin (Zocor)), cholesterol absorbers (e.g., ezetimibe (Zetia)), bile acid sequestrants (e.g., cholestyramine (Prevalite), colesevelam (Welchol), colestipol (Colestid)), ezetimibe-simvastatin (Vytorin), alirocumab (Praluent), evolocumab (Repatha), PKSK9 inhibitors, fibrates (e.g., fenofibrate (Tricor), gemfibrozil (Lopid)), niacin, phytosterols, and fish oils/omega 3 fatty acids, weight loss medications (orlistat (Xenical), lorcaserin (Belviq), phentermine and topiramate (Qsymia), buproprion and naltrexone (Contrave), liraglutide (Saxenda), phentermine, Adipex-P, Topamax, Desoxyn, Alli, Xenical, phendimetrazine, Tenuate, Fastin, Qsymia, bupropion, diethylpropion, HCG, methamphetamine, Bontril Slow Release, Didrex, lorcaserin, Saxenda, lonamin, Pregnyl, Bontril PDM, chorionic gonadotropin, phentermine/topiramate, Tagamet, Topiragen, Zantryl, liraglutide, T-Diet, Topamax Sprinkle, amphetamine, benzphetamine, bupropion/naltrexone, cimetidine, Evekeo, methylphenidate, Suprenza, Tenuate Dospan, Adipost, Atti-Plex P, Belviq XR, desvenlafaxine, Lomaira, Oby-Cap, Phendiet, Phentercot, Phentride, Prelu-2, Tagamet HB, Equaline Acid Reducter, Melfiat, Obezine, Phendiet-105, Recede, Regimex, Tepanil, Garcinia, Guarana, Hoodia, Ephedra), anticoagulants (Heparin, Warfarin, Apixaban, Dabigatran, Dalteparin, Edoxaban, Enoxaparin, Fondaparinux, Rivaroxaban, Betrixaban), Xa inhibitors, anti-fibrotics (nintedanib, pirfenidone, epinephrine, NSAIDs, antihistamines, corticosteroids (cortisone, predinsone), cyclophosphamide, azathioprine, micophenolate mofetil, N-acetylcysteine, proton pump inhibitors (Prilosec, Nexium), bronchodilators (albuterol, theophilline, ipratropium), mucolytics (guaifenesin, Dnase, N-acetylcysteine, hypertonic saline), anti-inflammatives (triamcinolone, flunisolide, fluticasone, beclomethasone, prednisone, methylprednisone, ibuprofen, montelukast, cromolyn, N-acetylcysteine), antibiotics (ciprofloxacin, co-trimoxazole, tobramycin, cephalexin, colistin, dicloxaccillin, azithromycin, amoxicillin, cipro, levofloxacin, piperacillin, ceftazidime, meropenem, amoxicillin/clav, piperacillin, meropenem), vitamins (ADEK, Fer-in-Sol, Polyviflor drops, Aquasol-A, Drisdol, Aquasol-E), pancreatic enzymes (pancrelipase, pancreatin), stool softeners (docusate, casanthranol, polyethylene glycol), GI drugs (omeprazol, ranitidine, metoclopramide), antihistamines (loratadine, cetirizine, fexofenadine), nasal sprays (Vancenase/Vanc AQ, Beconase/BecAQ, sinus rinse), silver sulfadiazine, santyl, urea, hibiclen, Silvadene, Biafine, Sarna, Venelex, Recedo, Rea LO, Luxamend, Bionect, Nuvail, Levicyn, Umecta, Dermasorb XM, Acticoat, Keragelt, Keragel, silver nitrate, mafenide, Sulfamylon, Polysporin, X-Viate, Umecta PD, Uramaxin, remeven, keratolytics, Neosporin, Bacitracin, Neomycin, Polymyxin, Bensal HP, Atrapro, Granulex, Vasolex, zinc paste, calamine, coal tar, ichthammol, pentoxifylline, iloprost, glyceryl trinitrate, calcium antagonists, corticosteroids, psoralen, phenytoin, retinoids, analgesics, colchicine, antiplatelets (aspirin), vasoconstrictors (nicotine, cocaine, adrenaline).

A compound of a fatty acid, such as an odd chain fatty acid, disclosed herein can be used in combination with one or more medical devices, medical treatments, or surgical treatments, e.g., surgical treatments for obesity (e.g., bariatric surgery such as gastric bypass surgery, laparoscopic adjustable gastric banding, biliopancreatic diversion with duodenal switch, gastric sleeve, vagal nerve blockade), catheter-directed thrombolysis, vena cava filter, venous thrombectomy, compression bandaging, vacuum assisted closure, intermittent pneumatic compression device, debridement (sharp, mechanical, autolytic (honey), enzymatic, or biosurgery (maggots)), ultraviolet light therapy, hyperbaric oxygen, radiant heat dressing, ultrasound therapy, laser, hydrotherapy, electrotherapy, electromagnetic therapy, and immunoglobulin replacement therapy.

### Dosing

As will be readily apparent to one skilled in the art, the useful in vivo dosage to be administered and the particular mode of administration will vary depending upon the age, weight, the severity of the condition, and mammalian species treated, the particular forms of the compounds employed, and the specific use for which these compounds are employed. The determination of effective dosage levels, that is the dosage levels necessary to achieve the desired result, can be accomplished by one skilled in the art using routine methods, for example, in vivo studies. Reference may be made to, for example, “Estimating the Maximum Safe Starting Dose in Initial Clinical Trials for Therapeutics in Adult Healthy Volunteers,” U.S. Food and Drug Administration, July 2005.

In some embodiments, a method provided herein may comprise administering a therapeutically effective amount of a composition provided herein. In some embodiments, a therapeutically effective amount may be determined by reference to the modulation of a marker of a condition associated with aging. In some embodiments, a therapeutically effective amount may be determined by reference to the modulation of a symptom of a condition provided herein. In still other embodiments, reference may be made to established guidelines for the conditions described herein, including, but not limited to, guidelines for the treatment of a condition provided herein including inflammation.

The dosage may vary broadly, depending upon the desired effects and the therapeutic indication, such as marker values. Alternatively, dosages may be based and calculated upon the surface area or weight of the patient, as understood by those of skill in the art. The exact dosage will be determined on a case-by-case basis, or, in some cases, will be left to the informed discretion of the subject. The daily dosage regimen for an adult human patient may be, for example, an oral dose of a fatty acid, such as an odd chain fatty acid or a very long even chain fatty acid, or a salt or derivative thereof, or a mixture of a plurality of fatty acids, or a salt or derivative thereof, from about 0.01 mg to about 10000 mg, from about 1 mg to about 5000 mg, from about 5 mg to about 2000 mg, from about 10 mg to about 1000 mg, or from about 50 mg to about 500 mg. A single dose may include a fatty acid, or a salt or derivative thereof, in about 0.01 mg, about 0.1 mg, about 1 mg, about 5 mg, about 10 mg, about 20 mg, about 50 mg, about 100 mg, about 200 mg, about 300 mg, about 400 mg, about 500 mg, about 600 mg, about 800 mg, about 900 mg, about 1000 mg, about 2000 mg, about 5000 mg, or more. The dosage may be adjusted according to the body mass of the subject, for example, the dosage may be about 0.001 mg/kg, about 0.01 mg/kg, about 0.1 mg/kg, about 0.5 mg/kg, about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 15 mg/kg, about 20 mg/kg, about 25 mg/kg, about 30 mg/kg, or higher. The dosage may be a single one or a series of two or more given in the course of one or more days, as is appropriate for the individual subject. In some embodiments, the compounds will be administered for a period of continuous therapy, for example for about a week or more (e.g., one week, two weeks, three weeks, four weeks, five weeks, six weeks, seven weeks, eight weeks, or more), for several weeks, for about a month or more (e.g., one month, two months, three months, four months, five months, six months, seven months, eight months, nine months, ten months, eleven months, twelve months, or more), for about a year or more, or for a plurality of years. In some embodiments, a fatty acid, such as an odd chain fatty acid or a very long even chain fatty acid, or a salt or derivative thereof, can be administered or ingested one time per day, two times per day, three times per day, or more.

As will be understood by those of skill in the art, in certain situations it may be necessary to administer the compounds disclosed herein in amounts that exceed the above-stated, preferred dosage range in order to effectively treat a subject.

Unit dosage forms can also be provided, e.g., individual packages with a premeasured amount of the composition, configured for administration on a predetermined schedule. Unit dosage forms configured for administration one to three times a day are preferred; however, in certain embodiments it may be desirable to configure the unit dosage form for administration more than three times a day, or less than one time per day.

Dosage amount and interval may be adjusted to the individual subject to provide plasma levels of the active moiety which are sufficient to maintain predetermined parameters, indicators, or marker values, or minimal effective concentration (MEC). Dosages necessary to achieve the desired result will depend on individual characteristics and route of administration. However, assays, for example, HPLC assays or bioassays, may be used to determine serum concentrations.

In some embodiments, the compounds and methods provided herein may be used in conjunction with devices and methods of using devices, for example, as provided in U.S. Pat. Nos. 7,651,845; 8,251,904; 8,251,904; 4,985,015; 8,827,957; 4,252,159; 5,318,521; 4,718,430; 9,713,600, 9,707,199, 9,687,461, 9,662,306, 9,561,206, U.S. Publ. No. 2011/0190702; U.S. Publ. No. 2017/0266144, U.S. Publ. No. 2016/0324814, U.S. Publ. No. 2016/0195559, U.S. Publ. No. 2016/0195558, U.S. Publ. No. 2016/0193172, 2 U.S. Publ. No. 016/0193171, U.S. Publ. No. 2016/0193170, WO 2016/111843, DE 2615061; and in conjunction with diagnostic devices, for example, as provided in U.S. Publ. No. 2012/0072236.

### Diagnosis and Monitoring

Provided herein are methods for the diagnosis and monitoring of conditions associated with aging.

In some embodiments, the method of diagnosis or monitoring may comprise the step of measuring a percentage of a fatty acid, such as an odd chain fatty acid or a very long even chain fatty acid, in a bodily fluid. In some embodiments, the method of diagnosis or monitoring may comprise the step of measuring a marker of a condition provided herein, including conditions associated with aging, in a subject. In some embodiments, the method of diagnosis or monitoring may comprise the step of measuring a marker of a condition associated with aging. In some embodiments, a correlation between one marker and another may prove instructive. In some embodiments, a condition associated with aging may be diagnosed by reference to a threshold level of erythrocyte sedimentation rate, for example, or serum odd chain fatty acid or serum very long even chain fatty acid. In some embodiments, a condition related to aging provided herein may be diagnosed by reference to a threshold level of a marker of the condition, for example, serum odd chain fatty acid percentage, serum concentration of an odd chain fatty acid, serum total odd chain fatty acid, serum very long even chain fatty acid, serum total very long even chain fatty acids, or a ratio between two serum fatty acids. For example, the threshold may be determined by reference to a symptom or marker of a condition associated with aging.

The percentage of a fatty acid, such as an odd chain fatty acid or a very long even chain fatty acid, or a marker of a condition associated with aging, in a subject may be monitored by any means. Samples for analysis may be derived any fluid or tissue of the subject. For example, from serum, plasma, erythrocyte membranes, urine, and feces.

### EXAMPLES

### Example 1

Aging is associated with a higher risk of hypercholesterolemia. It was hypothesized that oral administration of a synthetic odd chain saturated fatty acid would lower cholesterol in a traditional obese laboratory animal model.

This study examined the impact of daily oral administration of synthetic pentadecanoic acid (C15:0) on total and LDL cholesterol in an obese mouse model. Twenty C57BL/6J mice were fed a high fat diet (HFD) (D12492, 60% kcals fat) for 8 weeks. Study mice were then divided into the following two groups of ten: vehicle controls and pentadecanoic acid-treated (5 mg/kg body weight). The test article was a synthetic powder form stable at room temperature and purchased from Sigma-Aldrich (Products W433400 (≥99% C15:0). The test article was provided daily via gastric gavage for 12 weeks (84 days) while continuing ad libitum access to the HFD. Total cholesterol and low-density lipoprotein (LDL) cholesterol were measured at Day 0 and Day 84. Data from the treated group were compared to the control group using Wilcoxon rank sum analyses. Significance was defined as a P value less than or equal to 0.05.

Mice in the pentadecanoic acid treatment group tolerated the test article throughout the study. There were no early mortalities among mice in the treated group; one mouse in the control group had an unscheduled death on Week 7.

When comparing the pentadecanoic acid treatment group with the vehicle control group at Day 84, subjects treated with pentadecanoic acid (5 mg/kg) had lower total cholesterol and LDL cholesterol (Table 1). There were no differences in these variables between the two groups at Day 0 (data not shown).

Table 1 provides comparisons of cholesterol between obese mice treated with oral synthetic pentadecanoic acid for 84 days and controls.

TABLE 1

| | | Pentadecanoic Acid | Wilcoxon Two- |
|---|---|---|---|
| | HFD | Treatment Group | Sample Test |
| Index at Day 84 | Control | 5 mg/kg dose | P value |
| Total Cholesterol | 207 ± 20 | 183 ± 25 | 0.032 |
| (mg/dl) | | | |
| LDL Cholesterol | 10.7 ± 2.9 | 6.3 ± 3.6 | 0.036 |
| (mg/dl) | | | |

### Example 2

Aging is associated with a higher risk of hyperglobulinemia, fibrosis, thrombosis, and hypercholesterolemia. It was hypothesized that oral administration of a synthetic odd chain saturated fatty acid would lower serum globulins, cholesterol, and liver fibrosis, in a traditional laboratory animal model on a high fat diet.

This study examined the impact of daily oral administration of synthetic pentadecanoic acid (C15:0) on serum globulins, cholesterol, and liver fibrosis in an animal model fed a high fat diet. Sixteen New Zealand white rabbits were fed a high fat diet (HFD) (4% peanut oil and 0.5% cholesterol) for 2 weeks. Study rabbits were then divided into the following two groups of eight: high fat diet controls, and high fat diet animals treated with pentadecanoic acid (35 mg/kg body weight). The test article was a synthetic powder form stable at room temperature and purchased from Sigma-Aldrich (Products W433400 (≥99% pentadecanoic acid). The test article was provided daily in the diet for 11 weeks (77 days) while continuing ad libitum access to the HFD. Total cholesterol, platelets, and globulins were measured at Days 0 and 77.

On Day 77, liver tissues from HFD controls and pentadecanoic acid-treated animals were collected and scored histologically for fibrosis by a board-certified veterinary pathologist. Liver tissues were processed into paraffin-embedded blocks for H&E and picrosirius red staining for fibrosis (collagen deposition). Whole picrosirius red stained liver slides were scanned using Aperio ScanScope AT2 at 20×. Fibrosis stages were defined as follows: 0=none, 1=persinusoidal or periportal, 2=perisinusoidal and portal/periportal, 3=bridging fibrosis, 4=cirrhosis. Data from the treated group were compared to the control group using Wilcoxon rank sum analyses. Significance was defined as a P value less than or equal to 0.05.

Rabbits in the pentadecanoic acid treatment group tolerated the test article throughout the study. There were no early mortalities in any groups.

When comparing the pentadecanoic acid treatment group with the HFD control group at Day 77, subjects treated with pentadecanoic acid (35 mg/kg) had lower plasma cholesterol, platelets, and globulins. There were no differences in these variables among the two study groups at Day 0 (data not shown). Table 2 provides comparisons of plasma cholesterol, platelets, globulins, and creatine kinase between rabbits treated with oral synthetic pentadecanoic acid for 77 days and controls.

TABLE 2

| | | Pentadecanoic Acid | Wilcoxon Two- |
|---|---|---|---|
| | HFD | Treatment Group | Sample Test |
| Index at Day 84 | Control | 35 mg/kg dose | P value |
| Cholesterol (mg/dl) | 3576 ± 1018 | 2516 ± 746 | 0.024 |
| Globulins (G/dl) | 2.2 ± 0.3 | 1.3 ± 0.7 | 0.004 |

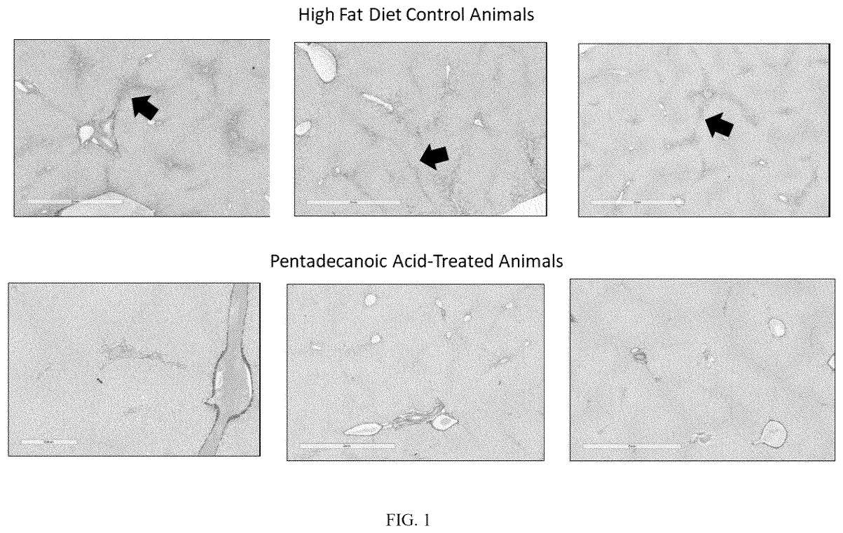

When comparing the pentadecanoic acid treatment group with the HFD control group at Day 77, subjects treated with pentadecanoic acid (35 mg/kg) had less severe liver fibrosis. FIG. 1 demonstrates the lack of Stage 3 bridging fibrosis in liver of rabbits treated with pentadecanoic acid. Table 3 provides comparisons of liver fibrosis stage scores between rabbits treated with oral synthetic pentadecanoic acid for 77 days and controls.

TABLE 3

| | | Pentadecanoic Acid | |
|---|---|---|---|
| | HFD | Treatment Group | Wilcoxon Two- |
| | Control | 35 mg/kg dose | Sample Test |
| Index at Day 84 | (n = 8) | (n = 8) | P value |
| Liver Fibrosis Stage | 2.9 ± 0.4 | 2.3 ± 0.5 | 0.016 |

### Example 3

Aging is associated with a higher risk of hyperglobulinemia, fibrosis, allergies, and cancer. It was hypothesized that primary human cells stimulated to mimic inflammatory, thrombotic, and fibrotic, responses would have reduced negative responses when exposed to a synthetic odd chain saturated fatty acid at appropriate concentrations.

This study examined the impact of synthetic pentadecanoic acid (C15:0) at four concentrations (740 nm and 2.2, 6.7, and 20 μM) on three primary human cell-based activity systems that were exposed to agents that mimicked allergic, thrombotic, and fibrotic disease states. In general, protein-based biomarkers in these systems were measured and compared with controls. Pentadecanoic acid was considered ‘active’ when 2 or more consecutive concentrations changed a given biomarker in the same direction relative to vehicle controls, were outside of the significance envelope of internal controls, and had at least one concentration with an effect size greater than 20% (log 10 ratio >0.1).

The first cell-based system recapitulated T cell-dependent activation of B cells that occurs in germinal centers to mimic allergy disease states. CD19⁺ B cells and peripheral blood mononuclear cells were stimulated with α-IgM and TCR ligands and incubated with pentadecanoic acid for 168 hours. In addition to controls that were not treated with pentadecanoic acid, a negative, non-stimulated system was also included. Secreted IgG was measured and compared with controls.

The second cell-based system recapitulated a Th1 inflammatory and prothrombotic environment. Primary endothelial cells were stimulated with IL-1β, TNF-α, and IFNγ and incubated with pentadecanoic acid for 24 hours. In addition to controls that were not treated with pentadecanoic acid, a negative, non-stimulated system was also included. Urokinase plasminogen activator receptor (uPAR) activation in this system was measured and compared with controls.

The third cell-based system recapitulated fibrosis and cell proliferation. Primary dermal fibroblasts were stimulated with IL-1β, TNFα, IFNγ, EGF, bFGF, and PDGF-BB and incubated with pentadecanoic acid for 24 hours. In addition to controls that were not treated with pentadecanoic acid, a negative, non-stimulated system was also included. Plasminogen activator inhibitor-1 (PAI-1), Collagen-I and fibroblast proliferation (at 72 hours) in this system were measured and compared with controls.

Pentadecanoic acid was non-cytotoxic in all three cell systems at all four concentrations (740 nm and 2.2, 6.7, and 20 μM).

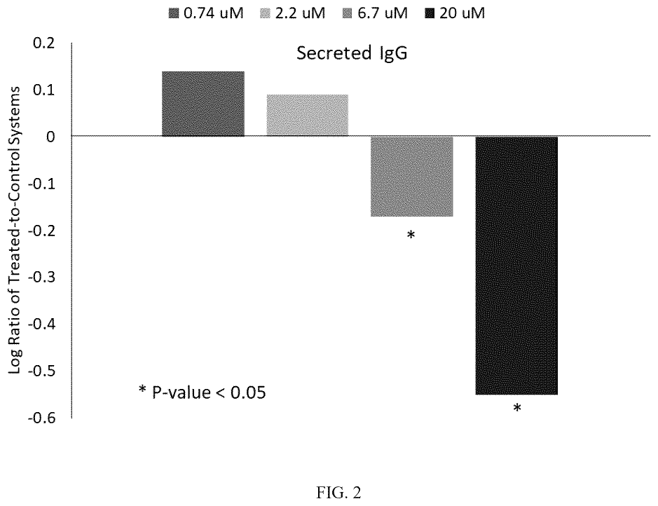

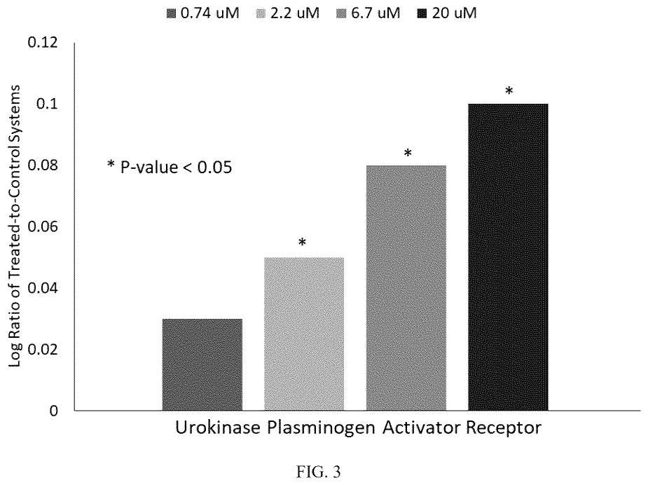

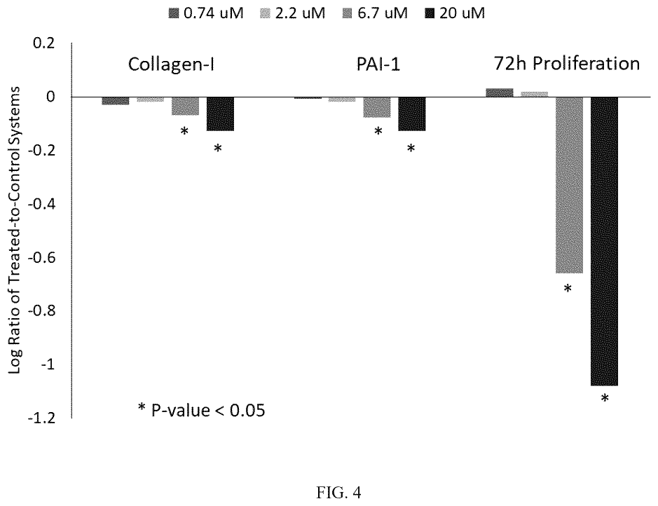

In these studies, cell-based systems mimicking inflammatory, prothrombotic, fibrotic, and cell proliferative responses were attenuated with pentadecanoic acid. FIGS. 2, 3 and 4 provide dose-dependent decreases in secreted IgG (FIG. 2), increases in uPAR (FIG. 3), and decreases in PAI-1, Collagen-I, and 72-hour fibroblast proliferation (FIG. 4) in human cell-based systems incubated with pentadecanoic acid. For all of these indices, pentadecanoic acid concentrations of 6.7 μM and 20 μM were bioactive.

Odd chain fatty acids (heptadecanoic acid, or C17:0, and pentadecanoic acid, or C15:0) are saturated fatty acids present in ruminant whole fat dairy products. Odd chain saturated fatty acids are assembled by bacteria in the rumen and pass from the rumen to the milk and have been used as biomarkers of dairy fat intake. Interestingly, despite consumer's movement away from high fat foods, dairy consumption, at times indicated by C15:0 and C17:0 blood levels, in humans has been associated with multiple health benefits, including a lower incidence of obesity and hypersensitivity disorders. To date, the mechanism of the benefits of dairy products on obesity and hypersensitivity has not been determined. Based upon the results using the methods of the embodiments, it can be proposed that odd chain saturated fatty acids may be key players in the anti-obesity and anti-hypersensitivity benefits of dairy products in humans.

To take advantage of these benefits, odd chain saturated fatty acids can be used in acid in a supplement, medical food, food additive, food fortifier, beverage additive, beverage fortifier, or pharmaceutical in any form, including as a tablet, encapsulated pill, gelcap pill, liquid suspension, spray, and powder. Additionally, diagnostic tests and assays for odd chain saturated fatty acids in human and animal samples (including blood (serum, plasma, and erythrocyte membranes), urine, and feces) can be used to detect low odd chain saturated fatty acids and to continually monitor odd chain saturated fatty acids levels in patients. The use of odd chain saturated fatty acids can prevent, stem, and treat: Aging and aging-associated conditions, including hypercholesterolemia, obesity, thrombosis, fibrosis, wound healing, hyperglobulinemia, hypersensitivity, and cancer cell proliferation and other related conditions.

Based on modified diet studies with dolphins, odd-chain saturated fatty acids have been proposed as a method to treat metabolic syndrome, diabetes, type 2 diabetes, elevated fasting plasma glucose, elevated serum triglycerides, hyperferritinemia, cardiovascular disease, fatty liver, abdominal obesity, proinflammatory state, elevation of C reactive protein, inflammation, anemia, insulin resistance, autoimmune disease, hypertension, cardiovascular disease, cancer, and neurodegenerative diseases, including Alzheimer's disease and other forms of dementia.

The data demonstrate a direct effect for odd chain saturated fatty acids on lowering cholesterol (total or LDL-cholesterol) and alleviating thrombosis, fibrosis, obesity, hyperglobulinemia, hypersensitivity and cancer cell proliferation. Obese mice on a high fat diet and concurrently treated with oral pentadecanoic acid (C15:0) daily for 12 weeks demonstrated lower cholesterol and lower percent body weight gain compared to control mice on the high fat diet alone. The cholesterol-lowering effects of pentadecanoic acid were repeated in our study with high fat diet-fed rabbits, in which rabbits fed a high fat diet and concurrently treated with daily oral pentadecanoic acid for 11 weeks had lower cholesterol compared to high fat diet controls. In addition, our high fat diet-fed rabbit study demonstrated the beneficial effects of pentadecanoic acid in lowering serum globulins and decreasing the severity of liver fibrosis compared to controls. These anti-hypersensitivity and anti-fibrotic effects in rabbits were repeated in our primary human cell phenotypic profile studies, including pentadecanoic acid's dose-dependent decreases in secreted immunoglobulin G from B cells, increases in urokinase plasminogen activator receptor in endothelial cells, and decreases in plasminogen activation inhibitor-1, Collagen-I, and dermal fibroblast proliferation.

The data demonstrate beneficial activity of odd chain saturated fatty acids at dose-dependent concentrations ranging from 2.2 μM to 20 μM, with most beneficial effects detected by at least 6.7 μM. Dosing of odd chain saturated fatty acids to achieve these serum, plasma, cell, or tissue levels is expected to confer the observed beneficial effects.

The following materials are incorporated herein by reference in the entirety: Venn-Watson S. (2015) Increased dietary intake of saturated fatty acid heptadecanoic acid (C17:0) associated with decreasing ferritin and alleviated metabolic syndrome in dolphins. PLOS ONE 10:e0132117.

Methods and compositions related to or applicable to aging or related conditions are discussed in the following references, which are incorporated by reference herein in their entirety: Yin, G. (2015) Pfeuffer M, Jaudszus A, Pentadecanoic and heptadecanoic acids: multifacted odd-chain fatty acids, Adv Nutr. 2016 Jul. 15; 7(4): 730-4; Fave, G. (2004) Physiochemical properties of lipids: new strategies to manage fatty acid bioavailability. Cell Bol Biol 50:815-831. Ramirez, M. (2001) Absorption and distribution of dietary fatty acids from different sources. Early Hum Develop 65: S95-S101; Jenkins B. (2015) A review of odd-chain fatty acid metabolism and the role of pentadecanoic acid (C15:0) and heptadecanoic acid (C17:0) in health and disease. Molecules 20:2425-44; Mansson H L (2008) Fatty acids in bovine milk fat. Food Nutr Res 52:4; Benatar J R. (2014) The effects of changing dairy intake on trans and saturated fatty acid levels—results from a randomized controlled study. Nutr J 13:32; Abdullah M M. (2015) Recommended dairy product intake modulates circulating fatty acid profile in healthy adults: a multi-centre cross-over study. Br J Nutr 113:435-44; Forouhi N. (2014) Differences in the prospective association between individual plasma phospholipid saturated fatty acids and incident type 2 diabetes: the EPIC-InterAct case-cohort study. Lancet Diabetes Endocrinol 2:810-8; Patel P. (2010) Fatty acids measured in plasma and erythrocyte-membrane phospholipids and derived by food-frequency questionnaire and the risk of new-onset type 2 diabetes: a pilot study in the European Prospective Investigation into Cancer and Nutrition (EPIC)-Norfolk cohort. Am J Clin Nutrition 92:1214-22; Krachler B. (2008) Fatty acid profile of the erythrocyte membrane preceding development of Type 2 diabetes mellitus. Nutrition, metabolism, and cardiovascular diseases. NMCD 18:503-10; Maruyama C. (2008) Differences in serum phospholipid fatty acid compositions and estimated desaturase activities between Japanese men with and without metabolic syndrome. J Atherscler Thromb 15:306-313; Choi H. (2005) Dairy consumption and risk of type 2 diabetes mellitus in men: a prospective study. JAMA Internal Med 165:997-1003; Kratz M. (2014) Dairy fat intake is associated with glucose tolerance, hepatic and systemic insulin sensitivity, and liver fat but not beta-cell function in humans. The American journal of clinical nutrition. 99:1385-96; Mennen L. (2000) Possible protective effect of bread and dairy products on the risk of the metabolic syndrome. Nutrition Research 20:335-47; Pereira M. (2002) Dairy consumption, obesity, and the insulin resistance syndrome in young adults: the CARDIA study. JAMA 287:2081-9; Sandrou D. (2000) Low-fat/calorie foods: current state and perspectives. Crit Rev Food Sci Nutr 40:427-47; Pfeuffer M. (2006) Milk and the metabolic syndrome. Obesity Rev 8:109-18; Mansson H L (2008) Fatty acids in bovine milk fat. Food Nutr Res 52:4; Fernandes R. (2013) Relationship between acute phase proteins and serum fatty acid composition in morbidly obese patients. Dis Markers 35:104-102; Forouhi N G. (2014) Differences in the prospective association between individual plasma phospholipid saturated fatty acids and incident type 2 diabetes: the EPIC-InterAct case-cohort study. Lancet Diabetes Endocrinol 2:810-818. doi: 10.1016/S2213-8587 (14) 70146-9 PMID: 25107467; Benatar JR. (2014) The effects of changing dairy intake on trans and saturated fatty acid levels-results from a randomized controlled study. Nutr J 13:32. doi: 10.1186/1475-2891-13-32 PMID: 24708591; Periera M A et al. (2002) Dairy consumption, obesity, and the insulin resistance syndrome in young adults: The CARDIA study. JAMA 287:2081-2089; Kratz M et al. (2013) The relationship between high-fat dairy consumption and obesity, cardiovascular, and metabolic disease. Eur J Nutr 52:1-24; Miyake Y et al. (2014) Maternal consumption of dairy products, calcium, and vitamin D during pregnancy and infantile allergic disorders. Ann Allergy Asthma Immunol 113:82-87; Kriesberg R A and Kasim S (1987) Cholesterol metabolism and aging, Am J Med 82:54-60; Anderson K M, Castelli W P, Levy D (1987) Cholesterol and mortality: 30 years of follow-up from the Framingham Study JAMA 257:2176-2180; Ghosh A K, Vaughan D E 2011 PAI-1 in tissue fibrosis J Cell Physiol 227:493-507; Hallgren et al. 1973 Lymphocyte phytohemagglutinin responsiveness, immunoglobulins and autoantibodies in aging humans. J Immunol 111:1101-1107.

### Example 4

In order to demonstrate that our screened compounds have activity relevant to multiple aging-associated diseases, human cell-based studies were completed using a selected subset of synthesized compounds. As a proof of concept, 14 compounds were selected from our compound library based on predicted efficacy in lowering inflammation in dolphins. Pure synthetic forms of these compounds were screened for cell-based activities (relevant to multiple aging-associated diseases and diseases that may impact longevity) using twelve different human primary cell systems mimicking various disease states (TABLE 4-description of primary human cell systems used to screen selected compounds in library).

### Methods

The Diversity PLUS panel allows test agent characterization in an unbiased way across a broad set of systems modeling various human disease states. These systems are designed to model complex human tissue and disease biology of the vasculature, skin, lung, and inflammatory tissues. Quantitative measurements of 148 biomarker activities across this broad panel, along with comparative analysis of biological activities from known bioactive agents, were used to predict and compare the efficacy and function of each selected compound at four concentrations (740 nm and 2.2, 6.7 and 20 μM).

BioMAP systems are constructed with one or more primary cell types from healthy human donors, with stimuli (such as cytokines or growth factors) added to capture relevant signaling networks that naturally occur in human tissue or pathological conditions. Vascular biology is modeled in both a Th1 (3C system) and a Th2 (4H system) inflammatory environment, as well as in a Th1 inflammatory state specific to arterial smooth muscle cells (CASM3C system). Additional systems recapitulate aspects of the systemic immune response including monocyte-driven Th1 inflammation (LPS system) or T cell stimulation (SAg system), chronic Th1 inflammation driven by macrophage activation (lMphg system) and the T cell-dependent activation of B cells that occurs in germinal centers (BT system). The BE3C system (Th1) and the BF4T system (Th2) represent airway inflammation of the lung, while the MyoF system models myofibroblast-lung tissue remodeling. Lastly, skin biology is addressed in the KF3CT system modeling Th1 cutaneous inflammation and the HDF3CGF system modeling wound healing.

Each test agent generates a signature BioMAP profile that is created from the changes in protein biomarker readouts within individual system environments. Biomarker readouts (7-17 per system) are selected for therapeutic and biological relevance, are predictive for disease outcomes or specific drug effects and are validated using agents with known mechanism of action (MoA). Each readout is measured quantitatively by immune-based methods that detect protein (e.g., ELISA) or functional assays that measure proliferation and viability. BioMAP readouts are diverse and include cell surface receptors, cytokines, chemokines, matrix molecules and enzymes. In total, the Diversity PLUS panel contains 148 biomarker readouts that capture biological changes that occur within the physiological context of the particular BioMAP system. Specific BioMAP activities have been correlated to in vivo biology, and multiparameter BioMAP profiles have been used to distinguish compounds based on MoA and target selectivity across diverse physiological systems.

Activated BioMAP systems were incubated with each compound for 24 to 72 hours. Protein-based biomarkers from activated cell systems were measured and compared with non-treated control systems. Biomarker activities were noted as ‘significant’ when at least one compound concentration was outside of the significance envelope and had an effect size >20% (log 10 ratio)>0.1. The BioMAP assays do not currently have cell systems to model metabolic diseases, including hyperinsulinemia, hyperglycemia, and dyslipidemia; thus, these assays were limited to assessing compounds' potential anti-inflammatory and antifibrotic properties.

Compounds included in this study included 10-undecanoate (conjugate base of 10-undecanoic acid; lipid, medium chain fatty acid), 10-heptanoate (conjugate base of 10-heptadecanoic acid; lipid, long chain fatty acid), and pentadecanoic acid (lipid, long chain fatty acid).

### Results

All compounds successfully demonstrated therapeutic activities, between concentrations of 0.7 and 20 μM, across multiple cell systems mimicking a variety of aging-associated diseases (TABLE 5-therapeutic activities of selected compounds in primary human cell systems mimicking various aging-associated diseases). Relevant cell-based disease systems that were successfully attenuated by our selected compounds represented Th1- and Th2-type inflammation, T-cell dependent B cell proliferation, allergy, asthma, atherosclerosis, autoimmunity, chronic inflammation, chronic obstructive pulmonary disease (COPD), Crohn's disease, cutaneous responses to tissue damage, fibrosis, hematological oncology, metabolic diseases, organ transplantation, psoriasis, pulmonary fibrosis, pulmonary responses to respiratory infections, restenosis, rheumatoid arthritis, sarcoidosis, stromal biology in tumors, systemic lupus erythematosus (SLE), ulcerative colitis, and vascular inflammation. Summary examples of diseases targeted by the selected compounds are provided (TABLE 6—Examples of diseases involving Th2 type inflammation or T-cell dependent B cell proliferation targeted by selected compounds, based on human primary cell phenotypic profiling activity data; TABLE 7—Examples of diseases involving Th1 type inflammation targeted by selected compounds, based on human primary cell phenotypic profiling activity data; TABLE 8—Examples of diseases targeted by selected compounds, based on human primary cell phenotypic profiling activity data).

Further, based on this study's findings, diseases that are driven or exacerbated by the following factors may be attenuated or treated by selected compounds: alpha smooth muscle actin (αSMA), CD40, CD69, collagen I, collagen III, decorin, e-selectin, eotaxin 3 (CCL26), fibroblast proliferation, human leukocyte antigen-DR isotype (HLA-DR), immunoglobulin G, interferon gamma-induced protein 10 (IP-10/CXCL10), interferon-inducible T cell alpha chemoattractant (I-TAC/CXCL11), interleukin (IL)-1, IL-la, IL-2, IL-6, IL-8 (CXCL8), IL-10, IL-17A, IL-17F, keratin 8/81, macrophage colony-stimulating factor (M-CSF), matrix metalloproteinase (MMP)-1, MMP-9, monocyte chemoattractant protein 1 (MCP-1), monokine induced by gamma interferon (MIG/CXCL9), plasminogen activation inhibitor 1 (PAI-1), prostaglandin E2 (PGE2), serum amyloid A, T or B cell proliferation, tissue plasminogen activator (tPA), tumor necrosis factor alpha (TNFα), vascular cell adhesion molecule (VCAM-1), vascular endothelial growth factor 2 (VEGFR2).

In summary, this study demonstrated that fatty acids across multiple pathways were identified in the dolphin metabolome and were predicted to have therapeutic activities relevant to aging-associated diseases, successfully demonstrated disease-modifying activities in human cell systems mimicking a variety of disease states.

TABLE 4

| Cell system | Human cell | | | |
|---|---|---|---|---|
| name | types | Stimulation | Disease/tissue relevance | System description |
| 4H | Venular | IL-4, | Th2-type inflammatory | Models Th2 type |
| | endothelial cells | histamine | conditions: autoimmunity, | vascular inflammation |
| | | | allergy, asthma, ulcerative | that promotes mast cell, |
| | | | colitis | basophil, eosinophil, T |
| | | | | and B cell recruitment |
| LPS | Venular | TLR4 | Chronic inflammatory | Models Th1 type |
| | endothelial | ligand | conditions where | chronic inflammation |
| | cells, peripheral | | monocytes play a key | and monocyte activation |
| | blood | | role: atherosclerosis, | process |
| | mononuclear | | restenosis, rheumatoid | |
| | cells | | arthritis, metabolic | |
| | | | diseases | |
| SAg | Venular | TCR ligands | T-cell driven | Models Th1 type |
| | endothelial | (1X) | inflammatory conditions | chronic inflammation |
| | cells, peripheral | | including organ | and T cell effector |
| | blood | | transplantation, | responses related to T |
| | mononuclear | | rheumatoid arthritis, | cell proliferation and |
| | cells | | psoriasis, Crohn's disease | activation |
| | | | and hematological | |
| | | | oncology | |
| BT | Peripheral blood | α-IgM, TCR | Diseases driven by B-cell | Models T cell |
| | mononuclear | ligands | activation and antibody | dependent B cell |
| | cells, B cells | (0.001X, | production: systemic | proliferation, activation |
| | | sub- | lupus erythematosus, | and class switching that |
| | | mitogenic | hematological oncology, | occurs in the germinal |
| | | levels) | autoimmune indications, | centers of secondary |
| | | | asthma and allergy | lymphoid organs |
| BF4T | Bronchial | IL-4, TNF-α | Allergy and asthma, | Models Th2 type lung |
| | epithelial cells, | | pulmonary fibrosis, | inflammation and |
| | dermal | | COPD exacerbations | environment that |
| | fibroblasts | | | promotes recruitment of |
| | | | | eosinophils, mast cells |
| | | | | and basal cells as well |
| | | | | as effector memory T |
| | | | | cells |
| BE3C | Bronchial | IL-1β, IFNγ, | Sarcoidosis and | Models Th1 type lung |
| | epithelial cells | TNFα | pulmonary responses to | inflammation and |
| | | | respiratory infections | environment that |
| | | | | promotes monocyte and |
| | | | | T cell adhesion and |
| | | | | recruitment |
| CASM3C | Coronary artery | IL-1β, | Chronic inflammatory | Models Th1 type vascular |
| | smooth muscle | TNFα, IFNγ | diseases, vascular | inflammation and |
| | cells | | inflammation and | environment that |
| | | | restenosis | promotes monocyte and |
| | | | | T cell recruitment |
| HDF3CGF | Dermal | IFNγ, | Various diseases | Models wound healing |
| | fibroblasts | TNFα, | including fibrosis, | and matrix/tissue |
| | | IL-1β, EGF, | rheumatoid arthritis, | remodeling in the context |
| | | bFGF, | psoriasis and stromal | of Th1-type inflammation |
| | | PDGF-BB | biology in tumors | |
| KF3CT | Keratinocytes, | IL-1β, | Cutaneous responses to | Models Th1 type |
| | dermal fibroblasts | IFNγ, | tissue damage caused | cutaneous inflammation |
| | | TGFβ, | by mechanical, | and environment that |
| | | TNFα | chemical or infectious | promotes monocyte and |
| | | | agents; certain states of | T cell adhesion and |
| | | | psoriasis and dermatitis | recruitment |
| MyoF | Lung fibroblasts | TGFβ, | Multiple fibrotic | Models general |
| | | TNFα | diseases | myofibroblast |
| | | | | differentiation and tissue |
| | | | | remodeling; readout |
| | | | | captures impacts on |
| | | | | translationally relevant |
| | | | | matrix remodeling, tissue |
| | | | | repair and inflammation |
| | | | | related to responses in |
| | | | | fibrotic tissue |
| /Mphg | Macrophages, | TLR2 | Inflammatory | Models Th1 type chronic |
| | venular | ligand | conditions where | inflammation and |
| | endothelial cells | | monocytes play a key | macrophage activation |
| | | | role including | responses |
| | | | atherosclerosis, | |
| | | | restenosis, rheumatoid | |
| | | | arthritis, and other | |
| | | | chronic inflammatory | |
| | | | conditions | |

TABLE 5

| | | | | | Significant log10 |
|---|---|---|---|---|---|
| | Human | | | Active | reduction of biomarkers in |
| | primary cell- | | Biomarkers significantly | concentrations | treated systems compared |
| Compound | based system | Disease systems | lowered by compound | (μM) | to nontreated controls |
| 10-heptadecanoate | BT | Asthma, oncology, | Secreted interleukin 2 | 6.7 | −0.11 |
| | | autoimmunity, allergy | (IL-2) | | |
| | BF4T | Fibrosis, lung | Eotaxin 3 (CCL26) | 2.2 | −0.10 |
| | | inflammation, asthma, | | 6.7 | −0.13 |
| | | allergy | | 20.0 | −0.31 |
| | CASM3C | Cardiovascular | Serum amyloid A | 2.2 | −0.13 |
| | | inflammation, restenosis | | | |
| | HDF3CGF | Fibrosis, chronic | Vascular cell adhesion | 6.7 | −0.17 |
| | | inflammation | molecule 1 (VCAM-1) | | |
| | | | Macrophage colony- | 6.7 | −0.1 |
| | | | stimulating factor (M-CSF) | | |
| 10-undecanoate | LPS | Chronic inflammation, | Secreted prostaglandin E2 | 0.7 | −0.11 |
| | | cardiovascular disease | (PGE2) | 2.2 | −0.08 |
| | | | | 6.7 | −0.20 |
| | | | CD40 | 20.0 | −0.11 |
| | BT | Asthma, oncology, | Secreted interleukin 17A | 2.2 | −0.25 |
| | | autoimmunity, allergy | (IE-17A) | | |
| | | | | 20.0 | −0.20 |
| | | | Tumor necrosis factor alpha | 20.0 | −0.12 |
| | | | (TNFα) | | |
| | SAg | Chronic inflammation, | CD40 | 0.7 | −0.07 |
| | | autoimmune disease | | 2.2 | −0.14 |
| | | | | 6.7 | −0.09 |
| | | | | 20.0 | −0.11 |
| | BE3C | COPD, lung inflammation | Tissue plasminogen activator | 2.2 | −0.08 |
| | | | (tPA) | 6.7 | −0.11 |
| | | | | 20.0 | −0.12 |
| | HDF3CGF | Fibrosis, chronic | Macrophage colony- | 0.7 | −0.11 |
| | | inflammation | stimulating factor (M-CSF) | 2.2 | −0.07 |
| | /Mphg | Chronic inflammation, | Secreted interleukin 10 | 20.0 | −0.21 |
| | | restenosis, cardiovascular | (IL-10) | | |
| | | disease | | | |
| Pentadecanoic acid | SAg | Chronic inflammation, | Monocyte chemoattractant | 6.7 | −0.18 |
| | | autoimmune disease | protein 1 (MCP-1) | 20.0 | −0.15 |
| | BT | Asthma, oncology, | Secreted immunoglobulin G | 6.7 | −0.17 |
| | | autoimmunity, allergy | (IgG) | 20.0 | −0.55 |
| | | | Secreted interleukin 17A | 2.2 | −0.25 |
| | | | (IL-17A) | 20.0 | −0.29 |
| | | | Secreted interleukin 17F | 2.2 | −0.16 |
| | | | (IL-17F) | 20.0 | −0.18 |
| | | | Secreted interleukin 6 (IL-6) | 20.0 | −0.29 |
| | HD3CGF | Fibrosis, chronic | Vascular cell adhesion | 20.0 | −0.28 |
| | | inflammation | molecule 1 (VCAM-1) | | |
| | | | Collagen I | 6.7 | −0.07 |
| | | | | 20.0 | −0.13 |
| | | | Collagen III | 20.0 | −0.13 |
| | | | Interferon gamma-induced | 20.0 | −0.14 |
| | | | protein 10 (IP-10)/CXCL10 | | |
| | | | Interferon-inducible T cell | 20.0 | −0.17 |
| | | | alpha chemoattractant (I- | | |
| | | | TAC)/CXCL11 | | |
| | | | Monokine induced by gamma | 20.0 | −0.10 |
| | | | interferon (MIG)/CXCL9 | | |
| | | | Plasminogen activation | 6.7 | −0.08 |
| | | | inhibitor 1 (PAI-1) | 20.0 | −0.13 |
| | | | 72-hour fibroblast | 6.7 | −0.67 |
| | | | proliferation | 20.0 | −1.1 |

TABLE 6

| | Significantly reduced cell-based biomarkers relevant to specific diseases and conditions | | | | | | | | | |
|---|---|---|---|---|---|---|---|---|---|---|
| | | T-cell | | | | | | | | |
| | Th2 | dependent | | | | | | | | |
| Clinical | type | Bcell | | | | Ulcera- | | | | Hemato- |
| compound | inflam- | prolif- | Auto- | | | tive | Pulmonary | | | logical |
| candidate | mation | eration | immunity | Allergy | Asthma | colitis | fibrosis | COPD | SLE | oncology |
| 10-heptadecanoate | x | x | x | x | x | | x | x | x | x |
| 10-undecanoate | | x | x | x | x | | | | x | x |
| Pentadecanoic acid | | x | x | x | x | | | | x | x |

TABLE 7

| Clinical | Significantly reduced cell-based biomarkers relevant to specific diseases and conditions | | | | | | | |
|---|---|---|---|---|---|---|---|---|
| compound | Th1 type | Chronic | Athero- | Reste- | Rheumatoid | Metabolic | Organ | |
| candidate | inflammation | inflammation | sclerosis | nosis | arthritis | diseases | transplantation | Psoriasis |
| 10-heptadecanoate | x | x | | x | x | | | x |
| 10-undecanoate | x | x | x | x | x | x | x | x |
| Pentadecanoic acid | x | x | | | x | | x | x |

TABLE 8

| | Significantly reduced cell-based biomarkers relevant to specific diseases and conditions | | | | | | | |
|---|---|---|---|---|---|---|---|---|
| | Cutaneous | | | | Pulmonary | | | |
| Clinical | responses | | | | responses to | | | Stromal |
| compound | to tissue | Crohn's | Hematological | | respiratory | Vascular | | biology in |
| candidate | damage | disease | oncology | Sarcoidosis | infections | inflammation | Fibrosis | tumors |

| | | | | | | | |
|---|---|---|---|---|---|---|---|
| 10-heptadecanoate | | | | | x | x | x |
| 10-undecanoate | x | x | x | x | | x | x |
| Pentadecanoic acid | x | x | | | | x | x |

### EXEMPLARY EMBODIMENTS

Method 1: A method of treatment or prophylaxis of a condition related to aging, comprising: administering, to a patient in need thereof, an effective amount of one or more odd chain fatty acids having from 9 carbon atoms to 31 carbon atoms or pharmaceutically acceptable salts thereof.

Method 2: Method 1, wherein the condition related to aging is selected from the group consisting of inflammation, anemia, hyperglycemia, dyslipidemia, hyperinsulinemia, liver disease, iron overload, impaired skin integrity, wound healing, scarring, pain, allergies, sleep disorders and problems, gastrointestinal disorders and problems, Th1-type inflammation, Th2-type inflammation, T-cell dependent B cell proliferation, allergy, asthma, atherosclerosis, autoimmunity, chronic inflammation, chronic obstructive pulmonary disease (COPD), Crohn's disease, cutaneous responses to tissue damage, fibrosis, hematological oncology, metabolic diseases, organ transplantation, psoriasis, pulmonary fibrosis, pulmonary responses to respiratory infections, restenosis, rheumatoid arthritis, sarcoidosis, stromal biology in tumors, systemic lupus erythematosus (SLE), ulcerative colitis, vascular inflammation, and diseases that are driven or exacerbated by one or more factors selected from the group consisting of alpha smooth muscle actin (αSMA), CD40, CD69, collagen I, collagen III, decorin, e-selectin, eotaxin 3 (CCL26), fibroblast proliferation, human leukocyte antigen-DR isotype (HLA-DR), immunoglobulin G, interferon gamma-induced protein 10 (IP-10/CXCL10), interferon-inducible T cell alpha chemoattractant (I-TAC/CXCL11), interleukin (IL)-1, IL-1α, IL-2, IL-6, IL-8 (CXCL8), IL-10, IL-17A, IL-17F, keratin 8/81, macrophage colony-stimulating factor (M-CSF), matrix metalloproteinase (MMP)-1, MMP-9, monocyte chemoattractant protein 1 (MCP-1), monokine induced by gamma interferon (MIG/CXCL9), plasminogen activation inhibitor 1 (PAI-1), prostaglandin E2 (PGE2), serum amyloid A, T or B cell proliferation, tissue plasminogen activator (tPA), tumor necrosis factor alpha (TNFα), vascular cell adhesion molecule (VCAM-1), and vascular endothelial growth factor 2 (VEGFR2).

Method 3: Any one of Methods 1 or 2, wherein the method is a method of prophylaxis.

Method 4: Any one of Methods 1 or 2, wherein the method is a method of treatment.

Method 5: Any one of Methods 1 through 4, wherein the condition related to aging is hypercholesterolemia.

Method 6: Any one of Methods 1 through 4, wherein the condition related to aging is selected from the group consisting of thrombosis, fibrosis, and poor wound healing.

Method 7: Method 6, wherein a marker or a symptom of thrombosis, fibrosis, or poor wound healing is modulated.

Method 8: Method 7, wherein the marker of thrombosis, fibrosis or poor wound healing is selected from the group consisting of serum, plasma, cell, or tissue levels of odd chain saturated fatty acids, urokinase plasminogen activator, plasminogen activation inhibitor-1, or collagen-I.

Method 9: Any one of Methods 1 through 4, wherein the condition related to aging is thrombosis.

Method 10: Any one of Methods 1 through 4, wherein the condition related to aging is selected from the group consisting of hyperglobulinemia and hypersensitivity disorders.

Method: Method 10, wherein a marker or a symptom of hyperglobulinemia or hypersensitivity is modulated.

Method 12: Method 11, wherein the marker of hyperglobulinemia or hypersensitivity is selected from the group consisting of serum, plasma, cell, or tissue levels of odd chain saturated fatty acids, serum globulins, or immunoglobulin G.

Method 13: Any one of Methods 1 through 4, wherein the condition related to aging is a hypersensitivity disorder.

Method 14: Any one of Methods 1 through 13, wherein a serum, plasma, or a red blood cell membrane concentration of the one or more odd chain fatty acids having from 9 carbon atoms to 31 carbon atoms is increased to a concentration greater than 2.2 μM and less than 30 μM.

Method 15: Any one of Methods 1 through 14, wherein the one or more odd chain fatty acids having from 9 carbon atoms to 31 carbon atoms is heptadecanoic acid.

Method 16: Any one of Methods 1 through 14, wherein the one or more odd chain fatty acids having from 9 carbon atoms to 31 carbon atoms is pentadecanoic acid.

Method 17: Any one of Methods 1 through 16, wherein a plurality of different odd chain fatty acids is administered.

Method 18: Any one of Methods 1 through 17, wherein a serum, plasma, red blood cell, or tissue concentration of the one or more odd chain fatty acids having from 9 carbon atoms to 31 carbon atoms is increased to greater than 2.2 μM and less than 30 μM.

Method 19: Any one of Methods 1 through 18, wherein the one or more odd chain fatty acids having from 9 carbon atoms to 31 carbon atoms or pharmaceutically acceptable salts thereof is provided as a pharmaceutical composition in a unit dosage form comprising the one or more odd chain fatty acids having from 9 carbon atoms to 31 carbon atoms or pharmaceutically acceptable salts thereof and a pharmaceutically acceptable carrier.

Method 20: Method 19, wherein the unit dosage form comprises from 0.01 mg to 10000 mg of the one or more odd chain fatty acids having from 9 carbon atoms to 31 carbon atoms or pharmaceutically acceptable salts thereof.

Method 21: Any one of Methods 19 through 20, wherein the pharmaceutical composition is substantially free from even chain saturated fatty acids.

Method 22: Any one of Methods 19 through 21, wherein the pharmaceutical composition is substantially free from polyunsaturated fatty acids.

Method 23: Any one of Methods 1 through 22, wherein the one or more odd chain fatty acids having from 9 carbon atoms to 31 carbon atoms or pharmaceutically acceptable salts thereof is administered to the patient once per day.

Pharmaceutical Composition 24: A pharmaceutical composition for treatment or prophylaxis of a condition related to aging, comprising: one or more fatty acids, or pharmaceutically acceptable salts thereof, wherein the one or more fatty acids are selected from the group consisting of odd chain saturated fatty acids; and a pharmaceutically acceptable carrier.

Pharmaceutical Composition 25: Pharmaceutical Composition 24, wherein the condition related to aging is selected from the group consisting of inflammation, anemia, hyperglycemia, dyslipidemia, hyperinsulinemia, liver disease, iron overload, impaired skin integrity, wound healing, scarring, pain, allergies, sleep disorders and problems, gastrointestinal disorders and problems, Th1-type inflammation, Th2-type inflammation, T-cell dependent B cell proliferation, allergy, asthma, atherosclerosis, autoimmunity, chronic inflammation, chronic obstructive pulmonary disease (COPD), Crohn's disease, cutaneous responses to tissue damage, fibrosis, hematological oncology, metabolic diseases, organ transplantation, psoriasis, pulmonary fibrosis, pulmonary responses to respiratory infections, restenosis, rheumatoid arthritis, sarcoidosis, stromal biology in tumors, systemic lupus erythematosus (SLE), ulcerative colitis, vascular inflammation, and diseases that are driven or exacerbated by one or more factors selected from the group consisting of alpha smooth muscle actin (αSMA), CD40, CD69, collagen I, collagen III, decorin, e-selectin, eotaxin 3 (CCL26), fibroblast proliferation, human leukocyte antigen-DR isotype (HLA-DR), immunoglobulin G, interferon gamma-induced protein 10 (IP-10/CXCL10), interferon-inducible T cell alpha chemoattractant (I-TAC/CXCL11), interleukin (IL)-1, IL-1α, IL-2, IL-6, IL-8 (CXCL8), IL-10, IL-17A, IL-17F, keratin 8/81, macrophage colony-stimulating factor (M-CSF), matrix metalloproteinase (MMP)-1, MMP-9, monocyte chemoattractant protein 1 (MCP-1), monokine induced by gamma interferon (MIG/CXCL9), plasminogen activation inhibitor 1 (PAI-1), prostaglandin E2 (PGE2), serum amyloid A, T or B cell proliferation, tissue plasminogen activator (tPA), tumor necrosis factor alpha (TNFα), vascular cell adhesion molecule (VCAM-1), and vascular endothelial growth factor 2 (VEGFR2)

Pharmaceutical Composition 26: Any one of Pharmaceutical Compositions 24 or 25, wherein the pharmaceutical composition is for treatment of a condition related to aging.

Pharmaceutical Composition 27: Any one of Pharmaceutical Compositions 24 or 26, wherein the pharmaceutical composition is for prophylaxis of a condition related to aging.

Pharmaceutical Composition 28: Any one of Pharmaceutical Compositions 24 through 27, wherein the condition related to aging is hypercholesterolemia.

Pharmaceutical Composition 29: Any one of Pharmaceutical Compositions 22 through 27, wherein the condition related to aging is selected from the group consisting of thrombosis, fibrosis, and poor wound healing.

Pharmaceutical Composition 30: Pharmaceutical Composition 29, adapted to modulate a marker or a symptom of thrombosis, fibrosis, or poor wound healing.

Pharmaceutical Composition 31: Pharmaceutical Composition 30, wherein the marker of thrombosis, fibrosis or poor wound healing is selected from the group consisting of serum, plasma, cell, or tissue levels of odd chain saturated fatty acids, urokinase plasminogen activator, plasminogen activation inhibitor-1, or collagen-I.

Pharmaceutical Composition 32: Any one of Pharmaceutical Compositions 24 through 27, wherein the condition related to aging is thrombosis.

Pharmaceutical Composition 33: Any one of Pharmaceutical Compositions 24 through 27, wherein the condition related to aging is selected from the group consisting of hyperglobulinemia and hypersensitivity disorders.

Pharmaceutical Composition 34: Pharmaceutical Composition 33, adapted for modulating a marker or a symptom of hyperglobulinemia or hypersensitivity.

Pharmaceutical Composition 35: Pharmaceutical Composition 34, wherein the marker of hyperglobulinemia or hypersensitivity is selected from the group consisting of serum, plasma, cell, or tissue levels of odd chain saturated fatty acids, serum globulins, or immunoglobulin G.

Pharmaceutical Composition 36: Any one of Pharmaceutical Compositions 24 through 27, wherein the condition related to aging is a hypersensitivity disorder.

Pharmaceutical Composition 37: Any one of Pharmaceutical Compositions 24 through 32, adapted to increase a serum, plasma, or a red blood cell membrane concentration of the one or more odd chain fatty acids having from 9 carbon atoms to 31 carbon atoms to a concentration greater than 2.2 μM and less than 30 μM.

Pharmaceutical Composition 38: Any one of Pharmaceutical Compositions 24 through 37, wherein the one or more odd chain fatty acids having from 9 carbon atoms to 31 carbon atoms is heptadecanoic acid.

Pharmaceutical Composition 39: Any one of Pharmaceutical Compositions 24 through 37, wherein the one or more odd chain fatty acids having from 9 carbon atoms to 31 carbon atoms is pentadecanoic acid.

Pharmaceutical Composition 40: Any one of Pharmaceutical Compositions 24 through 39, wherein a plurality of different odd chain fatty acids is administered.

Pharmaceutical Composition 41: Any one of Pharmaceutical Compositions 24 through 40, in a unit dosage form comprising the one or more odd chain fatty acids having from 9 carbon atoms to 31 carbon atoms or pharmaceutically acceptable salts thereof and a pharmaceutically acceptable carrier.

Pharmaceutical Composition 42: Any one of Pharmaceutical Compositions 24 through 41, wherein the unit dosage form comprises from 0.01 mg to 10000 mg of the one or more odd chain fatty acids having from 9 carbon atoms to 31 carbon atoms or pharmaceutically acceptable salts thereof.

Pharmaceutical Composition 43: Any one of Pharmaceutical Compositions 24 through 42, wherein the pharmaceutical composition is substantially free from even chain saturated fatty acids.

Pharmaceutical Composition 44: Any one of Pharmaceutical Compositions 24 through 43, wherein the pharmaceutical composition is substantially free from polyunsaturated fatty acids.

Pharmaceutical Composition 45: Any one of Pharmaceutical Compositions 24 through 44, in unit dosage form, adapted for administration of from 2.5 mg to 50 mg of the one or more odd chain fatty acids having from 9 carbon atoms to 31 carbon atoms or pharmaceutically acceptable salts thereof to the patient, per 1 kg of body weight, per day.

Pharmaceutical Composition 46: Any one of Pharmaceutical Compositions 24 through 45, in unit dosage form, adapted for administration to the patient once per day.

Use 47: Use of a pharmaceutical composition for treatment or prophylaxis of a condition related to aging, the pharmaceutical composition comprising: one or more fatty acids, or pharmaceutically acceptable salts thereof, wherein the one or more fatty acids are selected from the group consisting of odd chain saturated fatty acids; and a pharmaceutically acceptable carrier.

Use 48: Use 47, wherein the condition related to aging is selected from the group consisting of inflammation, anemia, hyperglycemia, dyslipidemia, hyperinsulinemia, liver disease, iron overload, impaired skin integrity, wound healing, scarring, pain, allergies, sleep disorders and problems, gastrointestinal disorders and problems, Th1-type inflammation, Th2-type inflammation, T-cell dependent B cell proliferation, allergy, asthma, atherosclerosis, autoimmunity, chronic inflammation, chronic obstructive pulmonary disease (COPD), Crohn's disease, cutaneous responses to tissue damage, fibrosis, hematological oncology, metabolic diseases, organ transplantation, psoriasis, pulmonary fibrosis, pulmonary responses to respiratory infections, restenosis, rheumatoid arthritis, sarcoidosis, stromal biology in tumors, systemic lupus erythematosus (SLE), ulcerative colitis, vascular inflammation, and diseases that are driven or exacerbated by one or more factors selected from the group consisting of alpha smooth muscle actin (αSMA), CD40, CD69, collagen I, collagen III, decorin, e-selectin, eotaxin 3 (CCL26), fibroblast proliferation, human leukocyte antigen-DR isotype (HLA-DR), immunoglobulin G, interferon gamma-induced protein 10 (IP-10/CXCL10), interferon-inducible T cell alpha chemoattractant (I-TAC/CXCL11), interleukin (IL)-1, IL-1α, IL-2, IL-6, IL-8 (CXCL8), IL-10, IL-17A, IL-17F, keratin 8/81, macrophage colony-stimulating factor (M-CSF), matrix metalloproteinase (MMP)-1, MMP-9, monocyte chemoattractant protein 1 (MCP-1), monokine induced by gamma interferon (MIG/CXCL9), plasminogen activation inhibitor 1 (PAI-1), prostaglandin E2 (PGE2), serum amyloid A, T or B cell proliferation, tissue plasminogen activator (tPA), tumor necrosis factor alpha (TNFα), vascular cell adhesion molecule (VCAM-1), and vascular endothelial growth factor 2 (VEGFR2)

Use 49: Any one of Uses 49 or 48, wherein the pharmaceutical composition is for treatment of a condition related to aging.

Use 50: Any one of Uses 47 or 48, wherein the pharmaceutical composition is for prophylaxis of a condition related to aging.

Use 51: Any one of Uses 43 through 46, wherein the condition related to aging is hypercholesterolemia.

Use 52: Any one of Uses 47 through 51, wherein the condition related to aging is selected from the group consisting of thrombosis, fibrosis, and poor wound healing.

Use 53: Use 52, wherein the pharmaceutical composition is adapted to modulate a marker or a symptom of thrombosis, fibrosis, or poor wound healing.

Use 54: Use 53, wherein the marker of thrombosis, fibrosis or poor wound healing is selected from the group consisting of serum, plasma, cell, or tissue levels of odd chain saturated fatty acids, urokinase plasminogen activator, plasminogen activation inhibitor-1, or collagen-I.

Use 55: Any one of Uses 47 through 51, wherein the condition related to aging is thrombosis.

Use 56: Any one of Uses 47 through 51, wherein the condition related to aging is selected from the group consisting of hyperglobulinemia and hypersensitivity disorders.

Use 57: Use 56, wherein the pharmaceutical composition is adapted for modulating a marker or a symptom of hyperglobulinemia or hypersensitivity.

Use 58: Any one of Uses 47 through 51, wherein the condition related to aging is a hypersensitivity disorder.

Use 59: Use 58, wherein the marker of hyperglobulinemia or hypersensitivity is selected from the group consisting of serum, plasma, cell, or tissue levels of odd chain saturated fatty acids, serum globulins, or immunoglobulin G.

Use 60: Any one of Uses 47 through 59, wherein the pharmaceutical composition is adapted to increase a serum, plasma, or a red blood cell membrane concentration of the one or more odd chain fatty acids having from 9 carbon atoms to 31 carbon atoms to a concentration greater than 2.2 μM and less than 30 μM.

Use 61: Any one of Uses 47 through 60, wherein the one or more odd chain fatty acids having from 9 carbon atoms to 31 carbon atoms is heptadecanoic acid.

Use 62: Any one of Uses 47 through 60, wherein the one or more odd chain fatty acids having from 9 carbon atoms to 31 carbon atoms is pentadecanoic acid.

Use 63: Any one of Uses 47 through 62, wherein a plurality of different odd chain fatty acids is administered.

Use 64: Any one of Uses 47 through 63, wherein the pharmaceutical composition is in a unit dosage form comprising the one or more odd chain fatty acids having from 9 carbon atoms to 31 carbon atoms or pharmaceutically acceptable salts thereof and a pharmaceutically acceptable carrier.

Use 65: Use 64, wherein the unit dosage form comprises from 0.01 mg to 10000 mg of the one or more odd chain fatty acids having from 9 carbon atoms to 31 carbon atoms or pharmaceutically acceptable salts thereof.

Use 66: Any one of Uses 47 through 65, wherein the pharmaceutical composition is substantially free from even chain saturated fatty acids.

Use 67: Any one of Uses 47 through 66, wherein the pharmaceutical composition is substantially free from polyunsaturated fatty acids.

Use 68: Any one of Uses 47 through 67, wherein the pharmaceutical composition is in unit dosage form, adapted for administration of from 2.5 mg to 50 mg of the one or more odd chain fatty acids having from 9 carbon atoms to 31 carbon atoms or pharmaceutically acceptable salts thereof to the patient, per 1 kg of body weight, per day.

Use 69: Any one of Uses 47 through 69, wherein the pharmaceutical composition is in in unit dosage form, adapted for administration to the patient once per day.

The above description presents the best mode contemplated for carrying out the present invention, and of the manner and process of making and using it, in such full, clear, concise, and exact terms as to enable any person skilled in the art to which it pertains to make and use this invention. This invention is, however, susceptible to modifications and alternate constructions from that discussed above that are fully equivalent. Consequently, this invention is not limited to the particular embodiments disclosed. On the contrary, this invention covers all modifications and alternate constructions coming within the spirit and scope of the invention as generally expressed by the following claims, which particularly point out and distinctly claim the subject matter of the invention. While the disclosure has been illustrated and described in detail in the drawings and foregoing description, such illustration and description are to be considered illustrative or exemplary and not restrictive.

All references cited herein are incorporated herein by reference in their entirety. To the extent publications and patents or patent applications incorporated by reference contradict the disclosure contained in the specification, the specification is intended to supersede and/or take precedence over any such contradictory material.

Unless otherwise defined, all terms (including technical and scientific terms) are to be given their ordinary and customary meaning to a person of ordinary skill in the art, and are not to be limited to a special or customized meaning unless expressly so defined herein. It should be noted that the use of particular terminology when describing certain features or aspects of the disclosure should not be taken to imply that the terminology is being re-defined herein to be restricted to include any specific characteristics of the features or aspects of the disclosure with which that terminology is associated. Terms and phrases used in this application, and variations thereof, especially in the appended claims, unless otherwise expressly stated, should be construed as open ended as opposed to limiting. As examples of the foregoing, the term ‘including’ should be read to mean ‘including, without limitation,’ ‘including but not limited to,’ or the like; the term ‘comprising’ as used herein is synonymous with ‘including,’ ‘containing,’ or ‘characterized by,’ and is inclusive or open-ended and does not exclude additional, unrecited elements or method steps; the term ‘having’ should be interpreted as ‘having at least;’ the term ‘includes’ should be interpreted as ‘includes but is not limited to;’ the term ‘example’ is used to provide exemplary instances of the item in discussion, not an exhaustive or limiting list thereof; adjectives such as ‘known’, ‘normal’, ‘standard’, and terms of similar meaning should not be construed as limiting the item described to a given time period or to an item available as of a given time, but instead should be read to encompass known, normal, or standard technologies that may be available or known now or at any time in the future; and use of terms like ‘preferably,’ ‘preferred,’ ‘desired,’ or ‘desirable,’ and words of similar meaning should not be understood as implying that certain features are critical, essential, or even important to the structure or function of the invention, but instead as merely intended to highlight alternative or additional features that may or may not be utilized in a particular embodiment of the invention. Likewise, a group of items linked with the conjunction ‘and’ should not be read as requiring that each and every one of those items be present in the grouping, but rather should be read as ‘and/or’ unless expressly stated otherwise. Similarly, a group of items linked with the conjunction ‘or’ should not be read as requiring mutual exclusivity among that group, but rather should be read as ‘and/or’ unless expressly stated otherwise.

Where a range of values is provided, it is understood that the upper and lower limit, and each intervening value between the upper and lower limit of the range is encompassed within the embodiments.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity. The indefinite article ‘a’ or ‘an’ does not exclude a plurality. A single processor or other unit may fulfill the functions of several items recited in the claims. The mere fact that certain measures are recited in mutually different dependent claims does not indicate that a combination of these measures cannot be used to advantage. Any reference signs in the claims should not be construed as limiting the scope.

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases ‘at least one’ and “one or more′ to introduce claim recitations. However, the use of such phrases should not be construed to imply that the introduction of a claim recitation by the indefinite articles ‘a’ or ‘an’ limits any particular claim containing such introduced claim recitation to embodiments containing only one such recitation, even when the same claim includes the introductory phrases ‘one or more’ or ‘at least one’ and indefinite articles such as ‘a’ or ‘an’ (e.g., ‘a’ and/or ‘an’ should typically be interpreted to mean ‘at least one’ or ‘one or more’); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of ‘two recitations,’ without other modifiers, typically means at least two recitations, or two or more recitations). Furthermore, in those instances where a convention analogous to ‘at least one of A, B, and C, etc.’ is used, in general such a construction is intended in the sense one having skill in the art would understand the convention (e.g., ‘a system having at least one of A, B, and C’ would include but not be limited to systems that have A alone, B alone, C alone, A and B together, A and C together, B and C together, and/or A, B, and C together, etc.). In those instances where a convention analogous to ‘at least one of A, B, or C, etc.’ is used, in general such a construction is intended in the sense one having skill in the art would understand the convention (e.g., ‘a system having at least one of A, B, or C’ would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase ‘A or B’ will be understood to include the possibilities of ‘A’ or ‘B’ or ‘A and B.’

All numbers expressing quantities of ingredients, reaction conditions, and so forth used in the specification are to be understood as being modified in all instances by the term ‘about.’ Accordingly, unless indicated to the contrary, the numerical parameters set forth herein are approximations that may vary depending upon the desired properties sought to be obtained. At the very least, and not as an attempt to limit the application of the doctrine of equivalents to the scope of any claims in any application claiming priority to the present application, each numerical parameter should be construed in light of the number of significant digits and ordinary rounding approaches.

Furthermore, although the foregoing has been described in some detail by way of illustrations and examples for purposes of clarity and understanding, it is apparent to those skilled in the art that certain changes and modifications may be practiced. Therefore, the description and examples should not be construed as limiting the scope of the invention to the specific embodiments and examples described herein, but rather to also cover all modification and alternatives coming with the true scope and spirit of the invention.

1-69. (canceled)

70. A method for treating stromal biology in tumors, scarring, wound healing, or allergies, comprising administering pentadecanoic acid, or a pharmaceutically acceptable salt thereof, to a subject in need thereof.

71. The method of claim 70, wherein the pentadecanoic acid, or a pharmaceutically acceptable salt thereof is in a pharmaceutical formulation.

72. The method of claim 71, wherein the pharmaceutical composition is in a unit dosage form.

73. The method of claim 72, wherein the pharmaceutical composition further comprises a pharmaceutically acceptable carrier.

74. The method of claim 71, wherein the unit dosage form comprises from 0.01 mg to 10000 mg of the pentadecanoic acid, or a pharmaceutically acceptable salt thereof.

75. The method of claim 71, wherein the pharmaceutical composition is substantially free from even chain saturated fatty acids.

76. The method of claim 71, wherein the pharmaceutical composition is substantially free from polyunsaturated fatty acids.

77. The method of claim 71, wherein the pharmaceutical composition is in unit dosage form, adapted for administration of from 2.5 mg to 50 mg of the pentadecanoic acid, or a pharmaceutically acceptable salt thereof to the patient, per 1 kg of body weight, per day.

78. A method of treating a condition related to aging, comprising:
administering one or more fatty acids, or pharmaceutically acceptable salts thereof, to a subject in need thereof,
wherein the condition related to aging is selected from the group consisting of inflammation, anemia, iron overload, impaired skin integrity, pain, gastrointestinal disorders and problems, Th1-type inflammation, Th2-type inflammation, T-cell dependent B cell proliferation, chronic inflammation, chronic obstructive pulmonary disease (COPD), Crohn's disease, cutaneous responses to tissue damage, fibrosis, hematological oncology, psoriasis, pulmonary fibrosis, pulmonary responses to respiratory infections, restenosis, rheumatoid arthritis, sarcoidosis, stromal biology in tumors, systemic lupus erythematosus (SLE), ulcerative colitis, vascular inflammation, and a disease that is driven or exacerbated by one or more factors selected from the group consisting of alpha smooth muscle actin (αSMA), CD40, CD69, collagen I, collagen III, decorin, e-selectin, eotaxin 3 (CCL26), fibroblast proliferation, human leukocyte antigen-DR isotype (HLA-DR), immunoglobulin G, interferon gamma-induced protein 10 (IP-10/CXCLI0), interferon-inducible T cell alpha chemoattractant (1-TAC/CXCLll), interleukin (IL)-1, IL-la, IL-2, IL-8 (CXCL8), IL-10, IL-17A, IL-17F, keratin 8/81, macrophage colony-stimulating factor (M-CSF), matrix metalloproteinase (MMP)-1, MMP-9, monokine induced by gamma interferon (MIG/CXCL9), plasminogen activation inhibitor I (PAI-I), prostaglandin E2 (PGE2), serum amyloid A, T or B cell proliferation, tissue plasminogen activator (tPA), vascular cell adhesion molecule (VCAM-1), and vascular endothelial growth factor 2 (VEGFR2).

79. The method of claim 78, wherein the condition related to aging is inflammation.

80. The method of claim 78, wherein the condition related to aging is COPD.

81. The method of claim 78, wherein the condition related to aging is stromal biology in tumors.

82. The method of claim 78, wherein the condition related to aging is pulmonary fibrosis.

83. The method of claim 78, wherein the condition related to aging is rheumatoid arthritis.

84. The method of claim 78, wherein the condition related to aging is SLE.

85. The method of claim 78, wherein the condition related to aging is psoriasis.

86. The method of claim 78, wherein the condition related to aging is sarcoidosis.

87. The method of claim 78, wherein the condition related to aging is restenosis.

88. The method of claim 78, wherein the condition related to aging is a disease that is driven or exacerbated by one or more factors selected from the group consisting of aSMA, CD40, CD69, collagen I, collagen III, decorin, e-selectin, CCL26, fibroblast proliferation, HLA-DR, immunoglobulin G, IP-10/CXCLI0, 1-TAC/CXCLll, IL-1, IL-la, IL-2, CXCL8, IL-10, IL-17A, IL-17F, keratin 8/81, M-CSF, MMP-1, MMP-9, MIG/CXCL9, PAI-I, PGE2, serum amyloid A, T or B cell proliferation, tPA, VCAM-1, and VEGFR2.

89. The method of claim 78, wherein the one or more fatty acids, or pharmaceutically acceptable salts thereof is pentadecanoic acid, or a pharmaceutically acceptable salt thereof.
